(19) United States
(12) Patent Application Publication
(10) Pub. No.: US 2017/0342129 A1
(43) Pub. Date: Nov. 30, 2017

(54) Protein Comprising a Collagen Domain of CCBE1 and Uses Thereof

(71) Applicant: Koninklijke Nederlandse Akademie van Wetenschappen, Amsterdam (NL)

(72) Inventors: M. Guy Roukens, Utrecht (NL); Stefan Schulte-Merker, Utrecht (NL); Dörte Schulte, Utrecht (NL)

(21) Appl. No.: 15/511,703

(22) PCT Filed: Sep. 18, 2015
(86) PCT No.: PCT/NL2015/050649

§ 371 Date: Jul. 19, 2017

(30) Foreign Application Priority Data
Sep. 19, 2014 (EP) 14185622.9

(51) Int. Cl. C07K 14/78; C07K 16/18; C12N 5/071; C07K 14/435; C07K 14/485; A61K 38/00

(52) U.S. Cl. CPC C07K 14/78; C07K 14/435; C07K 14/485; C07K 16/18; C12N 5/0602; A61K 38/00; C12N 2501/11; C12N 2501/998; C07K 2319/00

(57) ABSTRACT

The invention relates among others to a protein comprising a fragment of 30-240 amino acids of Collagen and calcium-binding EGF domain-containing protein 1 (CCBE1) having 0-10 amino acid substitutions with respect to the amino acid sequence of SEQ ID NO: 1, wherein said fragment comprises at least a collagen domain of CCBE1, or a variant thereof comprising 1, 2 or 3 amino acid substitutions. The invention further describes means and methods for the treatment of individuals with the protein or protein binding thereto.

The invention relates to the field of medicine. In particular the invention relates to the fields of lymphangiogenesis and cancer.

The lymphatic vasculature plays a key role in tissue fluid homeostasis, fat absorption and trafficking of immune cells¹. Dysfunction of lymphatic vessels leads to edema formation, and lymphatic vessels have also been implicated in a number of other pathological conditions such as inflammation and tumor metastasis².

In all vertebrates, lymphatic vessels originate mainly from endothelial cells in the cardinal vein. In mice, lymphatic endothelial cells (LECs) are first specified on the dorsal side of the cardinal vein by expression of the lymphatic endothelial marker Prox1³. Subsequently LECs migrate away as strings of loosely connected cells and reorganize into two large, lumenized lymphatics vessels, the dorso-laterally situated peripheral longitudinal lymphatic vessel (PLLV) and the more ventrally localized primordial thoracic duct (pTD)^{4,5}. From these structures, a lymphatic network emerges through sprouting lymphangiogenesis.

In zebrafish, lymphatic precursor cells arise when venous sprouts migrate dorsally from the cardinal vein at 32 hours post fertilization (hpf). Roughly half of these sprouts connect to an artery to form intersegmental veins, while the other half migrate dorsally to the horizontal myoseptum region and form a pool of lymphatic precursors cells, the parachordal lymphangioblasts (PLs)^{6,7}. These PLs subsequently migrate dorsally and ventrally along arteries to reorganize into the main lymphatic vessels, the TD and the dorsal longitudinal lymphatic vessels (DLLV) (reviewed in⁸).

The main lymphangiogenic signaling pathway involves VEGFC/VEGFR3 signaling. Both in mouse and in fish VEGFC/VEGFR3 signaling is induces the sprouting of venous cells from the cardinal vein^{6, 9, 10}. In VEGFC knock-out mice, LECs are specified, but fail to migrate away from the cardinal vein¹¹. The current model postulates that VEGFC acts as a morphogen to activate VEGFR3 signaling in LECs, which drives (future) LECs out of the cardinal vein. In Vegfc mutant zebrafish as well as in Vegfr3/Flt4 kinase dead mutants, venous sprouts do not migrate out of the cardinal vein and subsequently, the formation of PLs and the major lymphatic vessels (TD and DLLV) is impaired^{9, 12}. In humans, mutations in VEGFC or VEGFR3 have been linked to Milroy and Milroy-like disease, a form of hereditary primary lymphedema^{13, 14}.

Recently, Collagen and Calcium-binding EGF domains 1 (CCBE1) was identified to be essential for lymphatic development^{12, 15}. CCBE1 encodes a secreted protein that contains an EGF domain and a calcium-binding EGF domain at the N-terminus, and two collagen domains at the C-terminus. In zebrafish, Ccbe1 is required for lymphovenous sprouting from the cardinal vein. In CCBE1 knock-out mice, lymphatic progenitor cells are specified, but they do not migrate away from the CV, hence resembling the VEGFC phenotype. However, in contrast to VEGFC knock-out mice, where no lymphatic sprouts are formed, CCBE1 knock-out embryos display a distinct migratory effect and exhibit abnormal, short Prox1-positive sprouts which do not segregate from the cardinal vein E. In both zebrafish and mice, CCBE1 interacts genetically with VEGFC^{5, 10}. Recently, it has been shown that CCBE1 is a regulator of VEGFC processing and regulates Vegfc-mediated induction of Vegfr3 signaling during embryonic lymphangiogenesis^{10, 17}. In order for VEGFC to reach its full signaling capacity, the VEGFC protein needs to be activated by cleavage of the N- and C-terminal propeptides from the VEGF homology domain (VHD)¹⁶. In fact, CCBE1 is needed for activation of VEGFC by proteolytic processing via ADAMTS3¹⁷. Importantly, CCBE1 is linked to Hennekam syndrome (HS), a human recessive disease characterized by lymphedema, lymphangiectasia, and mental retardation¹⁸. To date, 9 different mutations in CCBE1 were identified to be causative¹⁹ for HS, with most mutations encoding missense mutations that affect the N-terminal part of the protein, namely the EGF domain, the Ca-binding EGF domain, or the cysteine sequence upstream of the EGF domain. Only two of the known mutations affect the collagen repeat domains, probably affecting the stability of a triple helix conformation of the collagen domain by altering conserved glycines in a Gly-X-Y motif¹³. One mutation introducing a frame-shift and encoding a predicted truncated protein lacking the collagen domain was found as a compound heterozygous mutation together with one of the other mutations.

Until now, it is unknown which parts of the CCBE1 protein are needed for its function. In the present invention the inventors identified functions of the different domains of CCBE1 using three different approaches. Function was elucidated in knock-in mice expressing deletion mutants of CCBE1. We found that some mutant Ccbe1 molecules were able to increase the activity of Vegfc in an in vivo zebrafish assay. Some mutant CCBE1 molecules can activate VEGFC processing in vitro. We found that deletion of a collagen domain inhibited CCBE1 function in vivo phenocopying CCBE1 knock-out mice. Furthermore, a CCBE1ΔCollagen mutant is not able to activate VEGFC signaling in vivo and in vitro. Surprisingly, however, we found that deletion of the EGF domains had no effect on VEGFC activation by CCBE1 in vitro and a CCBE1ΔEGF mutant is still at least partially active. In vivo CCBE1ΔEGF mice develop at least rudimentary lymphatics and therefore appear to have a less severe phenotype than CCBE1ΔCollagen mutant mice. Consistent with this, Ccbe1ΔEGF is still able to increase Vegfc signaling in zebrafish.

The invention discloses that the collagen domains of CCBE1 increase VEGFC activity in vivo and in vitro. The EGF domains are necessary for its full lymphangiogenic activity in vivo; however, they are dispensable for enhancement of VEGFC processing in vitro.

CCBE1 has been suggested to provide a therapeutic entry point for lymphedema treatment. The invention provides means and methods for therapeutic application.

### SUMMARY OF THE INVENTION

The invention provides a protein comprising a fragment of 30-240 amino acids of Collagen and calcium-binding EGF domain-containing protein 1 (CCBE1) having 0-10 amino acid substitutions with respect to the amino acid sequence of SEQ ID NO: 1, wherein said fragment comprises at least a collagen domain of CCBE1, or a variant thereof comprising 1, 2 or 3 amino acid substitutions.

The invention further provides a nucleic acid molecule encoding a protein of the invention.

Further provided is a cell or non-human animal comprising a nucleic acid molecule of the invention.

The invention further provides a binding protein that binds a collagen domain of a protein of the invention. The binding protein is preferably an antibody or a functional part, derivative and/or analogue thereof.

The invention further provides a method of producing lymphatic channels, increasing lymphangiogenesis and/or inducing the migration of lymphangioblasts in an animal, comprising administrating an effective amount of a protein of the invention, a nucleic acid molecule of the invention or a cell of the invention to said animal. The animal is preferably a human.

The invention further provides a method of inhibiting the formation of lymphatic channels, lymphangiogenesis and/or the migration of lymphangioblasts in an animal comprising administering an effective amount of a binding protein of the invention.

The invention further provides the protein, the nucleic acid molecule or the cell of the invention for use in producing lymphatic channels, increasing lymphangiogenesis and/or inducing the migration of lymphangioblasts in an animal, wherein the animal is preferably treated for cancer.

Further provided is the binding molecule of the invention, for use in inhibiting the formation of lymphatic channel, lymphangiogenesis and/or the migration of lymphangioblasts in an animal wherein the animal is preferably treated for cancer.

Also provided is a method for manipulating the formation of lymphatic channels, lymphangiogenesis and/or the migration of lymphangioblasts in a cell culture capable thereof, comprising providing the cell culture with the protein, the nucleic acid molecule, the cell, or the binding protein of the invention, or a combination thereof.

### DETAILED DESCRIPTION OF THE INVENTION

Representative sequences of ccbe1 can be found, for example, without limitation, in Genbank Accession Nos. NC_005117.2 (Rattus Norvegicus), NC_000018.8 (Homo Sapiens), NC_000084.5 (Mus Musculus), NC_006127.2 (Gallus Gallus). The list of sequences depicts a human Ccbe1 nucleotide and protein sequence.

Antibodies directed against Ccbe1 protein are commercially available (Novus Biologicals, Mouse anti Human). Methods for selecting antibodies with affinity for a molecule are also known. A skilled person can therefore produce antibodies against Ccbe1 protein and test such antibodies using this method.

With the term “animal” is meant any live animal, including free-living larval and/or reproducing larval forms, but excluding human fetal or human embryonic forms. Said animal can be any non-human animal which has a lymphatic system. In embodiments of the invention the animal is preferably a human. These embodiments are typically though not necessarily directed towards therapy. In embodiments the animal is preferably a non-human animal. Preferably said non-human animal is a vertebrate, because the lymphatic system of vertebrates is evolutionary more related to humans than lymphatic systems of other animals. Preferably said non-human animal is a mammal, more preferably a rodent or primate. Preferably a non-human primate. With the term “embryo” is meant an organism in the early stages of growth and differentiation. In embodiments encompassing an embryo or the commercial use thereof, the embryo is preferably a non-human embryo.

With the term “cell culture” is meant any in vitro culture of cells outside the body. Suitable cell cultures are HEK cultures, for instance to study VEGFC processing. Various other cultures are suitable such as cell cultures in which lymphangiogenesis is mimicked and include human cell cultures. Any in vitro-assay mimicking lymphangiogenesis can be used. Such assays are known in the art. For example, an in-vitro tube assay using lymphatic endothelial cells (LEC) and an invasion assay using LEC are described (Nakamura Cancer Sci (95); No. 1; p. 25-31(2004).

In a preferred embodiment, such in-vitro assay comprises an in vitro migration test. Preferably such in vitro migration test comprises placing cells (preferably LECs) into a chamber (Boyden chamber) that allows cells to move through a membrane (the bottom of the chamber) in response to a stimulus. An in vitro migration test is preferably based on a chamber of two medium-filled compartments separated by a microporous membrane. Typically, cells are placed in the upper compartment and are allowed to migrate through the pores of the membrane into the lower compartment, in which an agent is present. Preferably, after an appropriate incubation time, the membrane between the two compartments is fixed and stained, and the number of cells that have migrated to the lower side of the membrane is determined. Such in vitro migration test may also comprise a filter membrane migration assay or a trans-well migration assay. A number of different Boyden chamber devices are available commercially.

An in vitro migration test preferably comprises placing LECs into a Boyden chamber, induce migration with VEGFC, and determine whether knockdown of ccbe1 affects migration. In another preferred embodiment, such in vitro migration test comprises placing LECs on one side of the membrane, and provide a protein of the invention or, as control Ccbe1 protein on the other side of the membrane (preferably in the form of cells induced to express the protein of the invention or as a control Ccbe1). Preferably, migration or other behaviour of LECs is compared in the presence or absence of a protein of the invention or, as a control, Ccbe1 protein.

It is of course possible to add other cell types, such as tumour cells to such assay. Such assay with additional tumour cells can be used to screen compounds capable of inhibiting lymphangiogenesis in tumours. In a preferred embodiment said culture comprises a co-culture of two different types of cells. Preferably, at least one of said cell types comprises endothelial cells, preferably LEC cells. In a preferred embodiment, said other type of cells are cells expressing ccbe1. In a preferred embodiment, said endothelial cells and said ccbe1 are compatible, usually they are derived from the same animal species. Said two different types of cells are preferably but not necessarily from the same animal species. In one embodiment said other type of cells are HEK cells. Preferably said HEK cells comprise an expression cassette for the expression of a protein of the invention. In a preferred embodiment said endothelial cells are primate endothelial cells. Preferably said primate cells are human cells.

An expression cassette of the invention comprises a nucleic acid molecule comprising a nucleic acid molecule of the invention together with means for the expression of the amino acid sequence encoded by the nucleic acid molecule of the invention. Such means typically encompasses a promoter suitable for driving transcription operably linked to the nucleic acid molecule of the invention. Such means may further encompass other elements such as a transcription termination sequence, an intron etc.

With the term “lymphatic channel” is meant a vascular duct that carries lymph which is eventually added to the venous blood circulation. With the term “lymphangiogenesis” is meant the process of the formation of lymphatic channels. With the term “lymphangioblast” is meant a precursor cell capable of differentiation into a cell present in a lymphatic channel. Lymphangioblasts can be detected, among other means, by the presence of membrane bound marker, such as VEGF-C; VEGFR-3 or Prox1, or by the expression of stabilin1, tie2, lyve1 and/or fli1. With the term “migration” of a lymphangioblast is meant the process starting with budding of a lymphangioblast from a vein and migrating of said lymphangioblast into mesodermal tissue and ends when said lymphangioblast starts to contribute to lymphangiogenesis. Methods to detect migration of lymphatic endothelial cells are known in the art.

Methods for detecting lymphatic channels and cells in an animal are known to a skilled person. Lymphatic channels and lymphatic cells can be visualized by imaging. Detection can be done by using markers or labels specific for lymphatic channels, lymphatic cells or lymphatic endothelial cells. For example, antibodies directed against specific antigens can be used. A preferred method to determine whether the development of lymphatic channels or lymphangiogenesis is inhibited is by determining the presence of a thoracic duct or part thereof in a non-human animal or a non-human embryo. In a non-human mammal it is preferred to determine whether the development of lymphatic channels or lymphangiogenesis is inhibited in the ear. Inhibition can be measured by measuring swelling of the ear due to inadequate drainage of the lymph. Another method is measuring the drainage of a dye injected into the footpad of a rodent, away from the area of injection towards the lymph node.

The absence or reduction in the number of lymphatic channels, cells or lymphatic endothelial cells in said animal, embryo or cell culture is indicative for inhibition of development of lymphatic channels or lymphangiogenesis. With the term “inhibiting the development of lymphatic channels and/or lymphangiogenesis” is meant that the function of lymphatic vessels is impaired, and/or that the volume or the number of lymphatic channels or the number of cells involved in the process of lymphangiogenesis is significantly lowered in said animal, embryo or cell culture, compared to a control which has not been provided with said compound. Inhibition of the migration of a lymphangioblast is defined as an interference with the migration of a lymphangioblast, with a result that said lymphangioblast does not contribute to lymphangiogenesis. Preferably, inhibition between 70-100% is achieved. More preferably, between 80 and 100% is achieved. Even more preferably between 90 and 100%. Even more preferably between 95 and 100%. Most preferably said inhibition is between 97 and 100%.

In a preferred embodiment, said animal or said embryo is transparent, at least for the duration of the experiment. An advantage thereof is that this facilitates imaging of the development of lymphatic channels or lymphangiogenesis and/or the migration of lymphangioblasts.

The invention further provides a non-human animal comprising a nucleic acid of the invention. A person skilled in the art will know how to make such genetically modified non-human animals. Examples of for instance knockout procedures using cre/lox recombination systems can be found in Lakso et al. (1992) Proc. Natl. Acad. Sci. 89: 6232-36 and Pichel et al. (1993) Oncogene 8: 3333-42. Such non-human animals preferably do not express unmodified CCBE1 protein native to the species. Knockout animals can be generated using methods known in the art. Various CCBE1 knockout animals are known in the art.

A skilled person is familiar with several methods to induce expression of a protein. A preferred method for providing cells with a nucleic acid of the invention, or the capacity to express a protein of the invention is by means of a gene delivery vehicle comprising an expression cassette for expression of a nucleic acid or protein of the invention. In a preferred embodiment, said gene delivery vehicle comprises a adenoviral vector, an adeno-associated viral vector or a lentiviral viral vector. Preferably said gene delivery vehicle comprises a nucleic acid encoding a protein of the invention.

The non-human genetically modified animal of the invention preferably comprises a further genetic modification, wherein the modification comprises the transgenic expression of stabilin1, tie2, lyve1, flt4 and/or fli1 fused to a GFP or a derivative thereof. With the term “derivative” of GFP is meant any mutated form of GFP resulting in an improved functionality of GFP, for example blue fluorescent protein (EBFP, EBFP2, Azurite, mKalama1), cyan fluorescent protein (ECFP, Cerulean, CyPet), yellow fluorescent protein derivatives (YFP, Citrine, Venus, YPet) and BFP derivatives. An advantage thereof is that these markers facilitate the imaging of veins, and are useful when following migration of lymphangioblasts. Methods to produce animals with GFP constructs are known in the art. Examples of animals expressing tie2, lyve1, flt4 or fli1 GFP are also known in the art. Preferred genetically modified non-human animals encompass drosophila, mice, rats and fish preferably Danio Rerio.

In another aspect, the invention relates to a binding protein that binds a protein of the invention. The binding protein is preferably an antibody or a functional part, derivative and/or analogue thereof. The binding protein or antibody preferably binds a collagen domain of a protein of the invention. Preferably said collagen domain is the C-terminal collagen domain of CCBE1. CCBE1 in this embodiment is preferably human CCBE1. The binding protein or antibody is capable of inhibiting lymphangiogenesis. The binding protein binds the protein and inhibits CCBE1 function. With the term “antibody” is meant any immunoglobulin. Any immunoglobulin produced by any means or derived from any animal capable thereof can be used. With the term “functional part” of an antibody is meant a functional part of said antibody that comprises an antigen binding site. For example, Fab fragments can be used. The functional part, derivative or analogue retains the same kind binding properties of the antibody, not necessarily the same amount.

In another aspect, the invention relates to a pharmaceutical composition, comprising a protein, a nucleic acid molecule, a cell or binding molecule of the invention and a suitable pharmaceutical carrier or an adjuvant. Suitable pharmaceutical carriers or adjuvants for antibodies are well known in the art. A pharmaceutical carrier can be any compatible, non-toxic substance suitable for delivery of the compound to the patient. Sterile water, alcohol, fats, waxes, and inert solids may be included in the carrier. Pharmaceutically accepted adjuvants (buffering agents, dispersing agent) may also be incorporated into the pharmaceutical composition. The invention also provides the use of said composition in therapy.

In another aspect, the invention provides a method for treating an individual afflicted with cancer, comprising providing said individual in need thereof with a therapeutically effective amount of a protein, nucleic acid molecule, binding protein or cell of the invention. In another aspect, the invention provides a method for treating an individual afflicted with lymphedema, comprising providing said individual in need thereof with a therapeutically effective amount of a binding protein of the invention.

The lymphedema can be an acquired lymphedema or an inherited lymphedema. In one embodiment the lymphedema is a congenital lymph vessel disorder resulting in lymphedema. Congenital lymphedema results from having insufficient lymphatic vessels that they cannot handle all the lymph. Congenital lymphedema almost always affects the legs. Women are much more likely than men to have congenital lymphedema. Rarely, the swelling is obvious at birth. More often, the swelling appears later in life, as the volume of lymph increases and overwhelms the small number of lymph vessels. The swelling often starts gradually in one or both legs. A preferred congenital lymph vessel disorder for use of a method of the invention is selected from Meige, Nonne-Milroy and Hennekam syndrome. A most preferred lymph vessel disorder is Hennekam syndrome. Acquired lymphedema can be the result of surgery, the result of cancer trauma etc. Acquired lymphedema typically appears after major surgical treatment or irradiation, especially after cancer treatment. Lymph nodes and lymphatic vessels are surgically removed or ablated by radiation. For example, the arm tends to swell after removal of a cancerous breast and lymph nodes in the armpit. Scarring of lymphatic vessels from repeated infection also may cause lymphedema.

In another aspect, the invention relates to the use of an expression cassette according to the invention for the production of a medicament for the treatment or prevention of lymphedema. Lymphedema is a condition of localized fluid retention caused by a compromised lymphatic system. By providing an expression cassette capable of stimulating lymphangiogenesis, the effects of lymphedema can be effectively treated. The same treatment can also be used to prevent the onset of lymphedema formation.

In another aspect, the invention relates to a method for influencing the function of lymphatic vessels, the development of lymphatic channels, lymphangiogenesis and/or the migration of lymphangioblasts in an animal comprising administration of an effective amount of a protein of the invention, a binding molecule of the invention, a cell of the invention or an expression cassette of the invention to an animal.

The influence can be inhibition or stimulation of the development of lymphatic channels, lymphangiogenesis and/or the migration of lymphangioblasts. A binding molecule of the invention typically inhibits whereas a protein, a nucleic acid or cell of the invention typically stimulates.

The comparison of sequences and determination of percent identity and similarity between two sequences is well-known in the art and can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). When a position in a first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The protein of the invention comprises a fragment of 30-240 amino acids of Collagen and calcium-binding EGF domain-containing protein 1 (CCBE1) having 0-10 amino acid substitutions with respect to the amino acid sequence of SEQ ID NO: 1, wherein said fragment comprises at least a collagen domain of CCBE1, or a variant thereof comprising 1, 2 or 3 amino acid substitutions. The protein is capable of inducing lymph vessel formation. The protein can be a fragment of CCBE1 as such or comprise modifications, such as, e.g., stabilizing modifications. The protein may also include various tags, such as His, Myc, etc, as well as additional heterologous sequences. In one embodiment the protein is a fusion protein comprising the fragment of CCBE1 and one or more additional amino acids. The fusion protein can be a fusion of the fragment with an ortholog of human CCBE1, albeit that in such a case the fusion is does not contain an EGF domain, a calcium binding EGF domain, or a combination thereof. The protein is an inducer of lymphatic vessel formation. The protein will further be referred to as the “protein of the invention”, as opposed to “binding protein of the invention”, which term is reserved for a protein that binds to a protein of the invention.

The protein preferably has 30-500 amino acids, preferably 30-400, more preferably 30-300, more preferably 30-200, more preferably 30-100 amino acids. The protein can have more than one of said CCBE1 fragment. In one embodiment the protein comprises 2, 3, 4, 5 or 6 copies the fragment. Proteins comprising a repetition of one or both of the collagen domains are encompassed in the invention. The fragment preferably contains a continuous stretch of amino acids from CCBE1.

The CCBE1 fragment of can have 0-10 amino acid substitutions with respect to the amino acid sequence of SEQ ID NO: 1. An amino acid substitution, if any, is preferably a conservative amino acid substitution. In a preferred embodiment the CCBE1 fragment comprises 0-5 amino acid substitutions with respect to the amino acid sequence of SEQ ID NO: 1. In a preferred embodiment the CCBE1 fragment comprises 0-3 amino acid substitutions with respect to the amino acid sequence of SEQ ID NO: 1. More preferably the fragment does not contain amino acid substitution with respect to the amino acid sequence of SEQ ID NO: 1.

The fragment comprises at least a collagen domain of CCBE1, or a variant thereof comprising 1, 2 or 3 amino acid substitutions. The substitutions are preferably with respect to the sequence of the collagen domain in the sequence of SEQ ID NO: 1. Variants of said collagen domain preferably comprise 1 or 2 amino acid substitutions, more preferably 1 amino acid substitution. The sequence of the collagen domains is very well conserved in different species. It is preferred that the fragment comprises a collagen domain of CCBE1 without any amino acid substitutions with respect to the sequence of the collagen domain in SEQ ID NO: 1.

The number of amino acid substitutions in the fragment include amino acid substitutions in a variant as defined above. This implies that if the fragment contains a variant of a collagen domain with 3 amino acid substitutions, the remainder of the fragment does not contain more than 7 amino acid substitutions.

The comparison of sequences and determination of percent identity and similarity between two sequences is well-known in the art and can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). When a position in a first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The collagen domain in the protein preferably comprises amino acid 300-333 as defined in SEQ ID:NO 1 or a variant thereof comprising 1, 2 or 3 amino acid substitutions, or the equivalent of said domain in an ortholog of human CCBE1, or a variant thereof comprising 1, 2 or 3 amino acid substitutions. The fragment preferably comprises two or more collagen domains of CCBE1. The fragment preferably comprises two collagen domains. The two or more collagen domains can be a combination of the two collagen domains of CCBE1. It can also be a repetition of one collagen domain, or a combination comprising one or more of collagen domain A and one or more of collagen domain B as defined in SEQ ID NO: 1.

Collagen domain A in SEQ ID NO: 1 is present in amino acids 245-290 (inclusive). Collagen domain B in SEQ ID NO: 1 is present in amino acids 300-333 (inclusive).

The fragment comprising two or more collagen domains preferably comprises amino acid 245-290 as defined in SEQ ID:NO 1 or a variant thereof comprising 1, 2 or 3 amino acid substitutions; and amino acid 300-333 as defined in SEQ ID:NO 1 or a variant thereof comprising 1, 2 or 3 amino acid substitutions, or the equivalent in an ortholog of human CCBE1 or a variant of said equivalent comprising 1, 2 or 3 amino acid substitutions.

The ortholog is preferably a mammalian ortholog, more preferably a primate ortholog. The fragment can have an equivalent of one collagen domain, or equivalents of both collagen domains. The variants of the collagen domain are as defined herein above.

The fragment comprising two or more collagen domains preferably comprises amino acid 245-290 as defined in SEQ ID:NO 1; and amino acid 300-333 as defined in SEQ ID:NO 1.

In a preferred embodiment the fragment comprises amino acids 245-333 of human CCBE1 as defined in SEQ ID:NO 1 or a variant thereof comprising 1, 2, 3 or 4 amino acid substitutions with respect to the sequence as defined in SEQ ID NO: 1, or the equivalent thereof in an ortholog of human CCBE1 or a variant of said equivalent comprising 1, 2, 3 or 4 amino acid substitutions. The variant comprising 1, 2, 3 or 4 amino acid substitutions may comprise a collagen domain having 1, 2, or 3 amino acid substitutions as defined herein above. In such case the total number of amino acid substitutions of the fragment does not exceed 4.

The invention further provides a nucleic acid molecule encoding a protein of the invention. The nucleic acid molecule encoding the fragment of CCBE1, preferably comprises a nucleic acid sequence of the corresponding encoding region as depicted in SEQ ID: NO: 2. Various nucleic acid sequences can code for the same protein sequence as a result of the degeneracy of the genetic code. Different organisms have different codon preferences. In a preferred embodiment the sequence of the nucleic acid molecule is optimized for good expression in the organism or bacterium, fungus or yeast of interest. In a preferred embodiment the nucleic acid molecule is codon optimized for Escherichia, preferably E. coli. In another preferred embodiment the nucleic acid molecule is codon optimized for expression in yeast. In another preferred embodiment the nucleic acid molecule is codon optimized for expression in mammalian cells, preferably human cells, CHO cells or other mammalian cell lines developed for the large scale production of mammalian proteins.

The invention further provides a cell or non-human animal comprising a nucleic acid molecule of the invention. The cell or non-human animal is preferably a mammalian cell or non-human mammal.

The invention further provides a binding protein that binds a CCBE1 fragment in a protein of the invention. The binding protein preferably binds a collagen domain of a fragment in a protein of the invention. There are presently a number of proteins that can be tailored to bind specific proteins. Most commonly, however, antibodies are used. In the present invention the binding protein is preferably an antibody or functional part, derivative or analogue thereof.

The term “antibody” as used herein means a proteinaceous molecule, preferably belonging to the immunoglobulin class of proteins, containing one or more variable domains that bind an epitope on an antigen, where such domains are derived from or share sequence homology with the variable domain of an antibody. Antibodies for therapeutic use are preferably as close to natural antibodies of the subject to be treated as possible (for instance human antibodies for human subjects). Antibody binding can be expressed in terms of specificity and affinity. The specificity determines which antigen or epitope thereof is specifically bound by the binding domain. The affinity is a measure for the strength of binding to a particular antigen or epitope. Binding or specific binding, is defined as binding with affinities (KD) of at least 1×10e-5 6 M, more preferably 1×10e-7 M, more preferably higher than 1×10e-9 M. Typically, antibodies for therapeutic applications have affinities of up to 1×10e-10 M or higher. Antibodies such as mono and bispecific antibodies comprise the constant domains of a natural antibody. An antibody of the invention is typically a full length antibody, preferably of the human IgG subclass. An antibody of the present invention are is preferably of the human IgG1 or IgG4 subclass. Such antibodies are most commonly used in the clinic.

An antibody of the invention is preferably a “full length” antibody. The term ‘full length’ according to the invention is defined as comprising an essentially complete antibody, which however does not necessarily have all functions of an intact antibody. For the avoidance of doubt, a full length antibody contains two heavy and two light chains. Each chain contains constant (C) and variable (V) regions, which can be broken down into domains designated CH1, CH2, CH3, VH, and CL, VL. An antibody binds to antigen via the variable domains contained in the Fab portion, and after binding can interact with molecules and cells of the immune system through the constant domains, mostly through the Fc portion. The terms ‘variable domain’, ‘VH/VL pair’, ‘VH/VL’ are used herein interchangeably. Full length antibodies according to the invention encompass antibodies wherein mutations may be present that provide desired characteristics. Such mutations should not be deletions of substantial portions of any of the regions. However, antibodies wherein one or several amino acid residues are deleted, without essentially altering the binding characteristics of the resulting antibody are embraced within the term “full length antibody”. For instance, an IgG antibody can have 1-20 amino acid residue insertions, deletions or a combination thereof in the constant region. For instance, ADCC activity of an antibody can be improved when the antibody itself has a low ADCC activity, by slightly modifying the constant region of the antibody (Junttila, T. T., K. Parsons, et al. (2010). “Superior In vivo Efficacy of Afucosylated Trastuzumab in the Treatment of HER2-Amplified Breast Cancer.” Cancer Research 70(11): 4481-4489)

Full length IgG antibodies are preferred because of their favorable half life and the need to stay as close to fully autologous (human) molecules for reasons of immunogenicity. An antibody of the invention is preferably an IgG antibody, preferably a full length IgG1 antibody or IgG4 antibody. IgG1 is favored based on its long circulatory half life in man. In order to prevent any immunogenicity in humans it is preferred that the bispecific IgG antibody according to the invention is a human or humanized IgG. An antibody of the invention binds a collagen domain of CCBE1. It preferably binds collagen domain B of CCBE1 as defined in SEQ ID NO: 1. An antibody that binds a fragment of CCBE1 in a protein of the invention, preferably does not bind to another human protein. For the present invention the antibody is said not to bind to another human protein when it binds at least 100-fold less to any protein expressed in an adult human that is not CCBE1 and that also contains a collagen domain.

The invention further provides a method of enhancing lymphatic vessel function, producing lymphatic channels, increasing lymphangiogenesis and/or inducing the migration of lymphangioblasts in an animal, comprising administrating an effective amount of a protein, a nucleic acid molecule or a cell of the invention to said animal.

The invention further provides a method of inhibiting the formation of lymphatic channels, lymphangiogenesis and/or the migration of lymphangioblasts in an animal comprising administering an effective amount of a binding protein of the invention to said animal.

The invention further provides a method of the treatment of an individual that has cancer or is suspected of having cancer comprising administering an effective amount of a binding protein of the invention to said individual.

The invention further provides a binding protein of the invention for use in the treatment of an individual that has cancer or is suspected of having cancer.

The invention further provides, the protein, the nucleic acid molecule or the cell, of the invention or a combination thereof for use in enhancing lymphatic vessel function, in producing lymphatic channels, increasing lymphangiogenesis and/or inducing the migration of lymphangioblasts in an animal, wherein the animal is preferably treated for cancer.

The invention further provides the binding molecule of the invention, for use in inhibiting the formation of lymphatic channel, lymphangiogenesis and/or the migration of lymphangioblasts in an animal wherein the animal is preferably treated for cancer.

In methods of treatment or prevention or uses for the treatment or prevention as defined herein the animal is preferably a human.

The invention further provides, the protein, the nucleic acid molecule or the cell, of the invention or a combination thereof in uses and method described herein wherein in addition VEGFC activity is provided, administered or the like. VEGFC can be provided as protein, as a nucleic acid molecule encoding VEGFC to obtain expression thereof in a cell, or as a cell expressing VEGFC.

The invention further provides a method for manipulating the formation of lymphatic channels, lymphangiogenesis and/or the migration of lymphangioblasts in a cell culture capable thereof, comprising providing the cell culture with the protein, the nucleic acid molecule, the cell, the binding protein or a combination thereof.

All patent and literature references cited in the present specification are hereby incorporated by reference in their entirety.

The invention is further explained in the following examples. These examples do not limit the scope of the invention, but merely serve to clarify the invention.

### BRIEF DESCRIPTION OF THE DRAWINGS

FIG. 1: CCBE1 variants lacking the EGF domains or the collagen repeat domains cannot substitute for wild type CCBE1 in vivo.

(A) Schematic representation of CCBE1 wild type and deletion mutants. CCBE1ΔEGF and CCBE1ΔCol lack the EGF and Ca-EGF domains or both collagen repeat domains, respectively. Depicted are the signal peptide (SP), EGF and Ca-EGF domains, collagen repeat A (ColA) and collagen repeat B (ColB). (B) Genetic targeting strategy for the generation of CCBE1ΔEGF mice. Homologous recombination event replaces exons 4 and 5 with an FLPe flanked neomycin resistance cassette followed by a LoxP flanked exon 4 and 5. FLPe mediated deletion of the neomycin resistance cassette and Cre mediated deletion of exon 4 and 5 leads to a transcript which is depleted of the EGF and Ca-EGF domains. (C) Genetic targeting strategy used for generating CCBE1ΔCol and CCBE1FL mice. Homologous recombination replaces exon 7 with a construct containing exon 7 to 11 with or without the collagen repeats, followed by a transcriptional stop and an FLPe flanked neomycin resistance cassette. The neomycin cassette was removed by crossing heterozygous mice to FLPe mice. (D) Edema formation in CCBE1ΔEGF, CCBE1ΔCol and CCBE1LacZ mice at E14.5. CCBE1FL knock-in mice were indistinguishable from wild type controls.

FIG. 2: CCBE1ΔCollagen mice lack all lymphatic structures, while lumenized lymphatic structures do develop in CCBE1ΔEGF fetuses.

(A) Skin stainings of blood and lymphatic vessels of E14.5 fetuses visualized by PECAM-1 and Lyve-1/Prox1 staining. CCBE1ΔCol fetuses phenocopy CCBE1LacZ mice with a complete lack of lymphatic vessels in the skin. CCBE1ΔEGF skins feature remnant lymphatic structures which are unable to form a branched lymphatic network. CCBE1FL knock-in mice do not show any overt differences to wild type control fetuses. (B) Lyve-1 stainings of transverse paraffin sections of E14.5 fetuses showing lumenized lymphatic structures in CCBE1ΔEGF fetuses, but not in CCBE1ΔCol or CCBE1 LacZ fetuses. Note that lymphatic structures in CCBE1ΔEGF fetuses are smaller than in wild type control fetuses. Lymphatic structures in CCBE1FL knock-in mice are indistinguishable from those in control fetuses. CV: Cardinal vein, DA: Dorsal Aorta, LV: Lymphatic vessel, LS: Lymphatic structure, pTD: primordial Thoracic duct

FIG. 3: Migration of specified LECs from the cardinal vein is completely impaired in CCBE1ΔCollagen or CCBE1LacZ embryos, while lymphatic structures form in CCBE1ΔEGF embryos.

(A) 3D projections of whole mount immunostainings of cleared E12.5 embryos for Prox1, PECAM-1 and Lyve-1 analyzed on a confocal microscope. CCBE1ΔCol and CCBE1LacZ embryos do not show any LECs outside the cardinal vein, while CCBE1ΔEGF embryos exhibit remnant lymphatic structures at the position of the pTD and PLLV. CCBE1FL knock-in embryos do not show any overt defects. CV: Cardinal vein, LS: Lymphatic structure, pTD: primordial thoracic duct, PLLV: peripheral longitudinal lymphatic vessel

FIG. 4: CCBE1 lacking the EGF domains or the first collagen domain, but not CCBE1 lacking the second collagen domain can increase the activity of VEGFC in vivo.

(A-G) Injection of ccbe1 mRNA into Tg(shh:vegfc) induced aberrant, ectopic turning of intersegmental arteries at 32 hpf. Confocal projections show representative images of control and injected embryos in a Tg(fli1a:EGFP) background. In this assay, the ccbe1fof mutant has nearly full activity, while ccbe1ΔEGF and ccbe1ΔcolA have reduced activity compared to wt ccbe1. Removal of both collagen domains (ccbe1Δcol) or only the second collagen domain (ccbe1ΔcolB) completely abolishes the effect of ccbe1. (H) Percentage of embryos showing wild type (no arterial sprouting), mild (aberrant ISAs spanning up to two somites) or severe (aberrant ISAs spanning more than two somites) arterial sprouting in Tg(shh:vegfc) embryos that are uninjected or were injected with wt ccbe1, ccbe1fof, ccbe1ΔEGF, ccbe1Δcol, ccbe1ΔcolA and ccbe1ΔcolB (n=101, 427, 149, 182, 93, 149, 188, respectively). Injection of ccbe1 mRNA into wild type embryos does not induce strong bilateral sprouting of ISA (FIG. 9).

FIG. 5: The second collagen domain of CCBE1 is essential for activation of proteolytic processing of VEGFC in vitro.

(A) Schematic representation of CCBE1 deletion mutants. CCBE1ΔEGF, CCBE1ΔCol, CCBE1ΔColA and CCBE1ΔColB lack the EGF domains, both collagen domains or collagen domain A or B, respectively. Amino acids 291 to 299 comprising amino acids between the two collagen domains and amino acids 92 to 294 including both EGF domains and the first collagen repeat are deleted in CCBE1Δ291-299 and CCBE1 ΔEGFΔColA, respectively. Depicted are the signal peptide (SP), EGF and Ca-EGF domains, collagen repeat A (ColA) and collagen repeat B (ColB). (B-E) Conditioned medium from HEK-293T cells transfected with indicated constructs was collected two days after transfection and analyzed by Western blotting using antibodies for VEGFC and CCBE1. Note that VEGFC antibodies are able to recognize both the intermediate 31 kDa form and the fully processed mature 21 kDa form of VEGFC. CCBE1 antibodies recognize an epitope in the C-terminus of the protein, allowing detection of all variants used in this study. (B) Wt CCBE1 as well as CCBE1ΔEGF increased the amount of fully processed 21 kD VEGFC in the supernatant of HEK-293T cells, while CCBE1ΔCol had no effect on the levels of mature VEGFC. (C) CCBE1ΔColA and CCBE1Δ291-299 are able to increase the amounts of fully processed VEGFC in equal amounts as wt CCBE1, while CCBE1ΔColB had no effect on mature VEGFC levels. (D) Wt CCBE1, CCBE1ΔEGF and CCBE1ΔEGFColA, but not CCBE1ΔCol enhance VEGFC processing. (E) Expression of ADAMTS3 increases VEGFC processing which is further enhanced by co-expression of wt CCBE1, CCBE1ΔEGFbut not by CCBE1ΔCol.

FIG. 6: Effect of HS patient mutations on the activity of CCBE1.

(A) Schematic illustration of the functional domains of CCBE1 with an overview of the described mutations. Illustrated are the Signal Peptide (SP), EGF domain (EGF), Calcium binding EGF domain (Ca-EGF), collagen repeat A (ColA), and collagen repeat B (ColB). (B) Injection of ccbe1-myc mRNA and Ccbe1-mycR150C into Tg(shh:vegfc) embryos leads to strong bilateral sprouting of ISA while ccbe1-myc G313R has nearly no effect on arterial sprouting. (C) Quantification of the percentage of embryos with wild type (no arterial sprouting), mild (aberrant ISAs spanning up to two somites) or severe (aberrant ISAs spanning more than two somites) arterial sprouting in Tg(shh:vegfc) embryos upon injection of ccbe1-myc, ccbe1-mycR150C or ccbe1-mycG327R mRNA. n=94, 178, 137, 113 for uninjected controls, ccbe1-myc, ccbe1-mycR158 and ccbe1-G327R, respectively. (D) VEGFC processing assays were performed as in FIG. 5 B-E. Wt CCBE1 and CCBE1 R158C stimulate processing of VEGFC, while CCBE1 G327R has only strongly reduced activity.

FIGS. 7A and 7B: Model for regulation of VEGFC by CCBE1

Schematic model of CCBE1 domains interacting with ECM components and the ADAMTS3 protease, respectively. The N-terminal part of CCBE1 binds to the ECM, thereby restricting the range of CCBE1 activity and (ultimately) determining the site of VEGFC processing. The collagen repeat domains activate ADAMTS3 and are crucial (especially the Col B domain) for the eventual processing step which produces fully mature VEGFC protein. The EGF domains are indispensable in vivo for creating local high-points of VEGFC production, while in vitro (possibly due to high expression levels of transfected CCBE1 variants) they are not required. The collagen repeat domains are essential in both settings. Thus, CCBE1 generates locations of high VEGFC activity which directs LECs via VEGFR3 signaling.

FIG. 8: CCBE1ΔEGF and CCBE1ΔCol cannot substitute for wild type CCBE1 in vivo.

(A) Sequencing of cDNA isolated from homozygous CCBE1ΔEGF embryos showing correct splicing from exon 3 to 6. (B) Edema formation in CCBE1ΔEGF/CCBE1ΔCol fetuses at E14.5.

FIG. 9: Rudimentary lymphatics develop in the skin of CCBE1ΔEGF, but not of CCBE1ΔCollagen fetuses.

Skin stainings of blood and lymphatic vessels visualized by PECAM-1 and Lyve-1/Prox1 staining in E16.5 embryos. CCBE1ΔCol fetuses phenocopy CCBE1LacZ mice with a complete lack of lymphatic vessels in the skin.CCBE1ΔEGF skins feature remnant lymphatic structures which are unable to form a branched lymphatic network. CCBE1FL knock-in mice do not show any overt differences to wild type control fetuses.

FIG. 10: CCBE1 lacking the EGF domains or the first collagen domain, but not CCBE1 lacking the second collagen domain can increase the activity of VEGFC in vivo.

Percentage of embryos showing wild type (no arterial sprouting), mild (aberrant ISAs spanning up to two somites) or severe (aberrant ISAs spanning more than two somites) arterial sprouting in Tg(shh:vegfc) and wild type embryos that are uninjected or were injected with the indicated ccbe1 variant mRNAs. Tg(shh:vegfc) embryos: n=101, 427, 149, 182, 93, 149, 188 for injection of wt ccbe1, ccbe1fof, ccbe1ΔEGF, ccbe1Δcol, ccbe1ΔcolA and ccbe1Δcol mRNA, respectively. Wt embryos: n=55, 117, 42, 53, 52, 69, 56 for injection of f wt ccbe1, ccbe1fof, ccbe1ΔEGF, ccbe1Δcol, ccbe1ΔcolA and ccbe1Δcol mRNA, respectively

FIG. 11: A D170E mutation in CCBE1 equivalent to the fof mutation in zebrafish does not affect CCBE1 function in vitro.

The VEGFC processing assay was performed as in FIG. 5 (B-E). CCBE1 D170E activates processing of VEGFC similar to wt CCBE1 and CCBE1ΔEGF, while CCBE1ΔCol had no effect on VEGFC processing.

### EXAMPLES

Material and Methods

Mouse Lines:

Mouse lines were maintained at the Hubrecht Institute using standard husbandry conditions. Experiments were approved by the local Animal Experimentation Committee (DEC). Generation of CCBE1ΔEGF, CCBE1ΔCol and CCBE1FL mice is described herein below. All mouse lines were crossed at least 5 times to C57Bl6 mice before analysis.

Zebrafish Lines

The Tg(fli1a:EGFP) and Tg(shh:vegfc) were described previously^{10, 20}.

mRNA Injection

ccbe1 mRNA was transcribed from a NotI-linearised template using SP6 RNA polymerase and the mMessage mMachine Kit (Ambion), and injected at 400 pg/embryo.

Whole Mount Staining of Murine Fetal Skin

Whole mount staining of dorsal skin preparations of fetuses from staged matings was performed as previously described²¹ using anti-PECAM1, anti-Lyve-1 and anti-Prox1 as primary antibodies. Images were acquired on a Leica SP8 confocal microscope.

Staining of Mouse Fetuses

Paraffin sections were dewaxed and rehydrated. After blocking of endogenous peroxidase in blocking buffer (0.040M citric acid, 0.121M disodium hydrogen phosphate, 0.030M sodium azide, 1.5% hydrogen peroxide) for 15 min at room temperature, epitope retrieval in citrate buffer (10 mM sodium citrate monohydrate, pH 6.0) and blocking in 2% BSA in PBS for 1 h, tissue sections were incubated over night at 4° C. with an anti-Lyve-1 antibody followed by incubation with the secondary antibody added (polymer HRP-labelled anti-mouse/rabbit, Envision) for 30 min at room temperature. Peroxidase was detected by DAB substrate using the SIGMAFAST DAB with Metal Enhancer Tablet Set according to manufacturer's instructions. Tissue sections were counterstained with Mayer's haematoxylin.

Whole Mount Staining and Optical Clearing of Mouse Embryos

Whole mount embryos were stained and cleared as described previously²² using anti-Prox1, anti-PECAM-1 and anti-Lyve-1 as primary antibodies. Cleared embryos were imaged on a Leica SP8 confocal microscope. 3D stacks were generated using Fiji (Image J).

VEGFC Processing In Vitro

VEGFC processing in vitro was performed as described a. Briefly, HEK293T cells were transfected with VEGC and/or CCBE1 cDNA using XtremeGene (Roche) according to manufacturer's instructions. Conditioned media were collected and mixed with Laemmli sample buffer. Western blotting analysis was performed using VEGFC (VEGFC isoform 103 antibody, antibodies-online) and CCBE1 (HPA041374, Sigma) antibodies.

Antibodies and Reagents:

The following antibodies were used: Anti-Lyve1 (#11-034, Angiobio), Anti-Lyve-1 (AF2125, R&D Systems), Anti-CD31 Mec13.3 (#550274, Pharmingen), anti-Prox1 (102-PA32, ReliaTech), anti-Prox1 (AF2727, R&D Systems), anti-VEGFC (isoform 103 antibody, antibodies-online), and anti-CCBE1 (HPA041374 Sigma). All Alexa-coupled antibodies were purchased from Invitrogen, HRP coupled antibodies were used from Dako.

Constructs:

Ccbe1 (wild type), ccbe1fof, ccbe1-myc, ccbe1-mycR150 and ccbe1myc-G313R cDNA clones have been described previously^{12, 18}. Zebrafish ccbe1ΔEGF was generated by amplification of ccbe1 pCS2 followed by religation. Zebrafish ccbe1Δcol, ccbe1ΔcolA and ccbe ΔcolB were generated by site-directed mutagenesis using wt ccbe1 in PCS2 as template. Human CCBE1 was subcloned into the pBabe vector in Xho1-EcoR1 restriction sites. Mutant variants of CCBE1 for expression in human cells were generated by site directed mutagenesis. Primer sequences used for generation of deletion mutants and mutants containing the HS mutations are given in Table I.

TABLE I

| Construct | Primer forward (5′-3′) | Primer reverse (5′-3′) |
|---|---|---|
| Zebrafish Ccbe1 ΔEGF | ttctggaatacactgaccc | gaaaaatcagacaacgtcatg |
| Zebrafish ccbe1Δcol | atgacgacaaattcattttcctttgacttcctgtta | taacaggaagtcaaaggaaaatgaatttgtcgtcat |
| Zebrafish ccbe1ΔcolA | atgacgacaaattcattttcccctgacctgtcgcat | atgcgacaggtcaggggaaaatgaatttgtcgtcat |
| Zebrafish ccbe1ΔcolB | ctgtcgcatatcaaacagtcctttgacttcctgtta | taacaggaagtcaaaggactgtttgatatgcgacag |
| Human CCBE1 | ctcgagatggtgccgccgcctccg | gaattcctatgggtagaagtctct |
| Human CCBE1 D170E | tacatccgggaagatgaagggaagacatgtacc | ggtacatgtcttcccttcatcttcccggatgta |
| Human CCBE1 ΔEGF | atcccagaagattacgacaggggagacaaatatccc | gggatatttgtctcccctgtcgtaatcttctgggat |
| Human CCBE1 Δcol | ctggcctcaaacacctactctttcgacttcctgcta | tagcaggaagtcgaaagagtaggtgtttgaggccag |
| Human CCBE1 ΔcolA | ctggcctcaaacacctactctcctgatctgtcccac | gtgggacagatcaggagagtaggtgtttgaggccag |
| Human CCBE1 ΔcolB | ctgtcccacattaagcaatctttcgacttcctgcta | tagcaggaagtcgaaagattgcttaatgtgggacag |
| Human CCBE1 4291-299 | atgggacccatgggaccaggccggaggggccctgtg | cacagggcccctccggcctggtcccatgggtcccat |
| Human CCBE1 ΔEGFΔcolA | ccagaagattacgactcccacattaagcaa | ttgcttaatgtgggagtcgtaatcttctgg |
| Human CCBE1 R158C | accttgggcagctactgctgcgagtgccgggaa | ttcccggcactcgcagcagtagctgcccaaggt |
| Human CCBE1 G327R | gcgcctgggcccagaaggtctccaggaccccct | agggggtcctggagaccttctgggcccaggcgc |

Targeting Strategy for the Generation of CCBE1ΔEGF and CCBE1ΔCol Mice:

CCBE1ΔEGF mice were generated by homologous recombination of mouse SV129 ES cells using a targeting vector that contains a 4.2 kb long arm of homology 5′ of exon 4, an FLPe flanked neomycin resistance cassette, a LoxP flanked exon 4 to 5 and a 2 kb short arm of homology 3′ of exon 5. Neomycin-resistant clones were screened by Southern blot analysis. The Neomycin resistance cassettes were removed by crossing to FLPe mice, and exon 4 and 5 of CCBE1 were deleted by crossing floxed mice to PGK-Cre mice. For the generation of CCBE1ΔCol mice, mouse SV129 ES cells were transfected using a targeting vector comprising a 4.5 kb arm of homology 5′ of exon 7, the cDNA of exon 7 to 11 lacking bp encoding aa 248 to 337 followed by a Flag-HA tag, a transcriptional stop, an FLPe flanked neomycin resistance cassette followed by a single LoxP site and a 4.2 kb arm of homology 3′ of exon 7. CCBE1FL mice were generated analogous to CCBE1ΔCol mice encoding the complete cDNA of exon 7 to 11. The Neomycin resistance cassette was removed by crossing to FLPe mice.

Results

CCBE1 Variants Lacking Either the EGF or the Collagen Repeat Domains Cannot Substitute for Wild Type CCBE1 In Vivo.

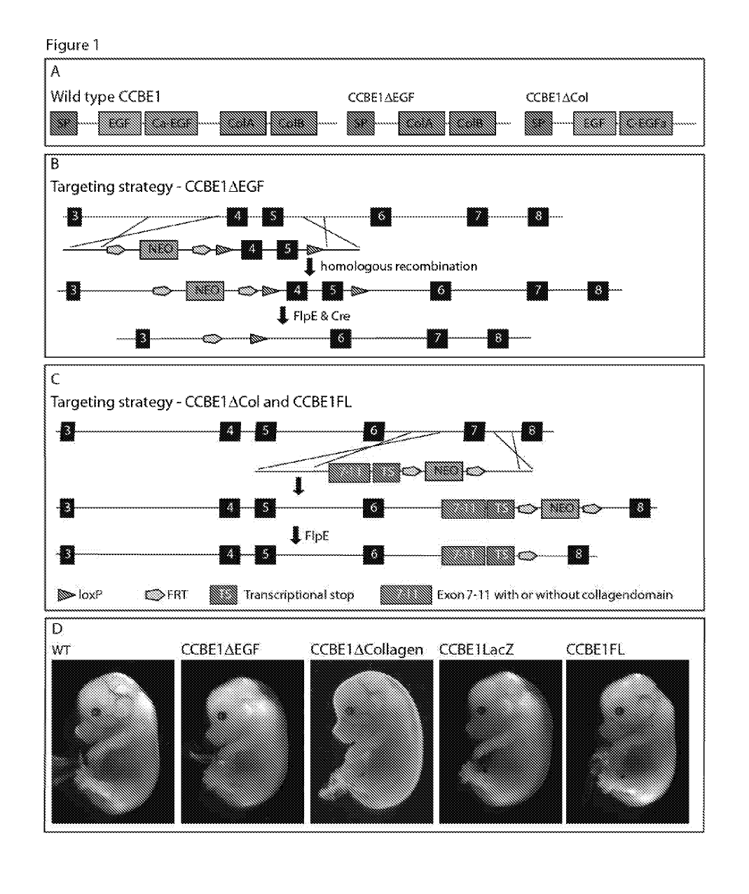

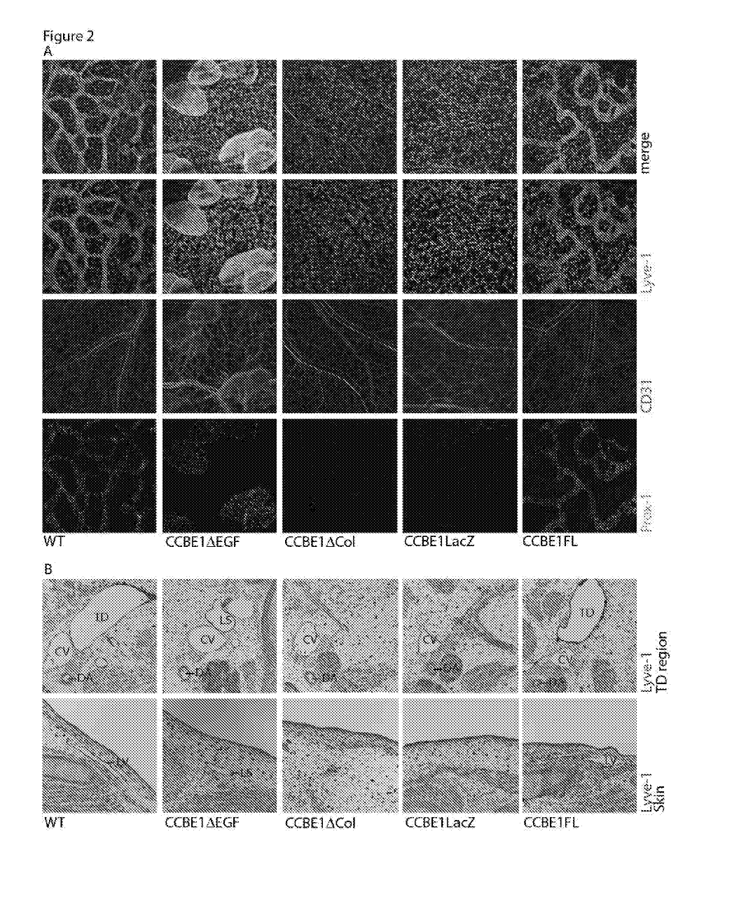

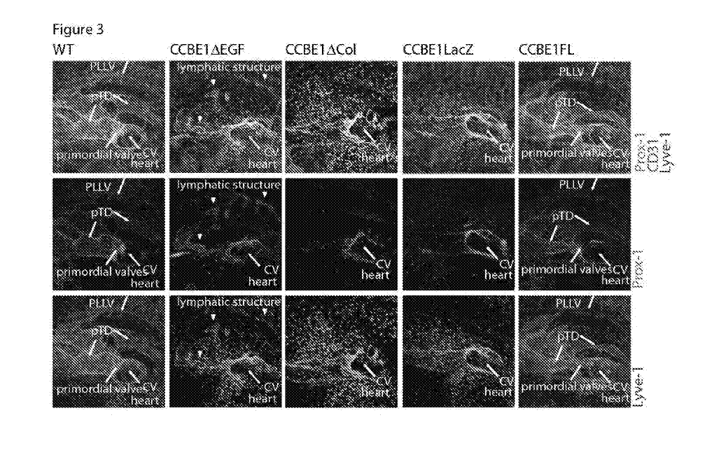

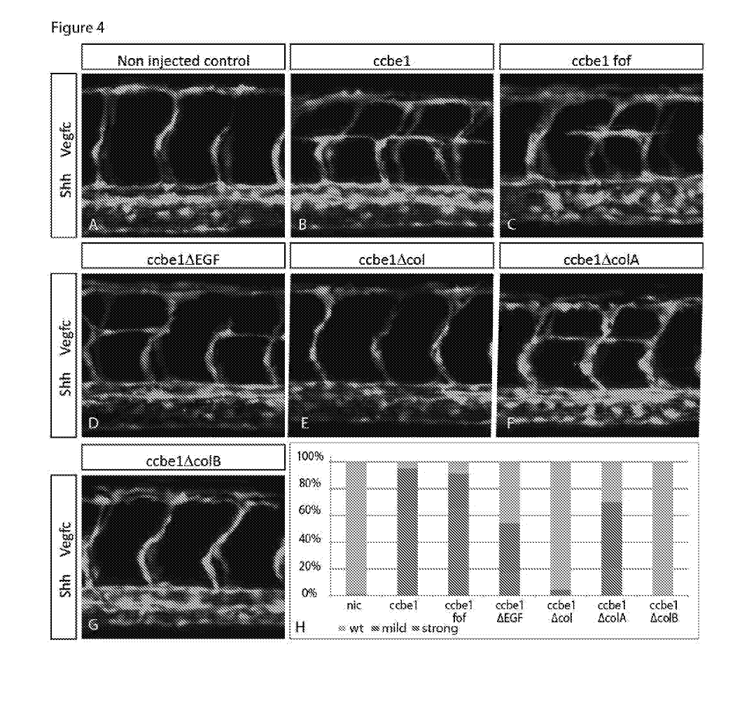

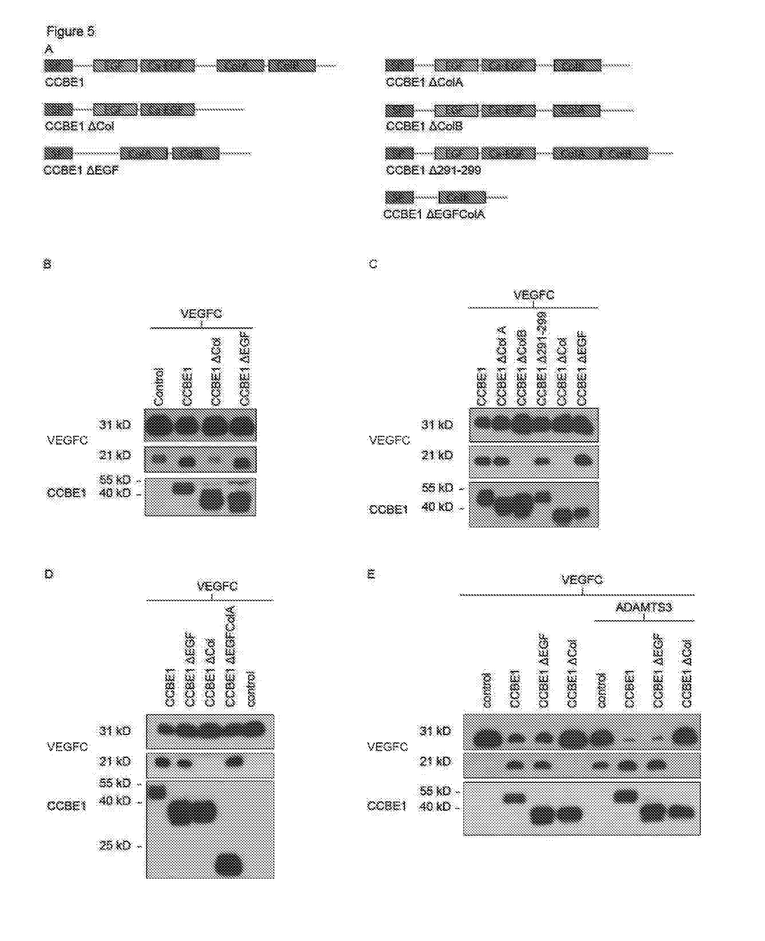

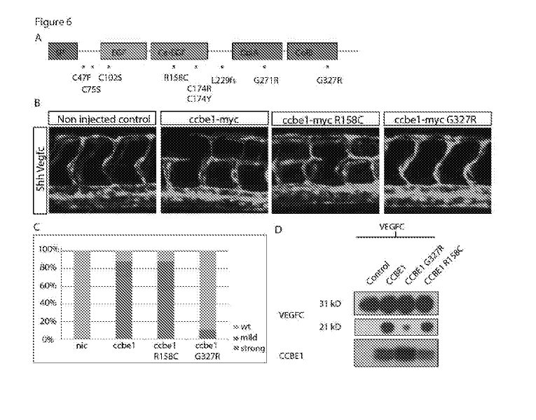

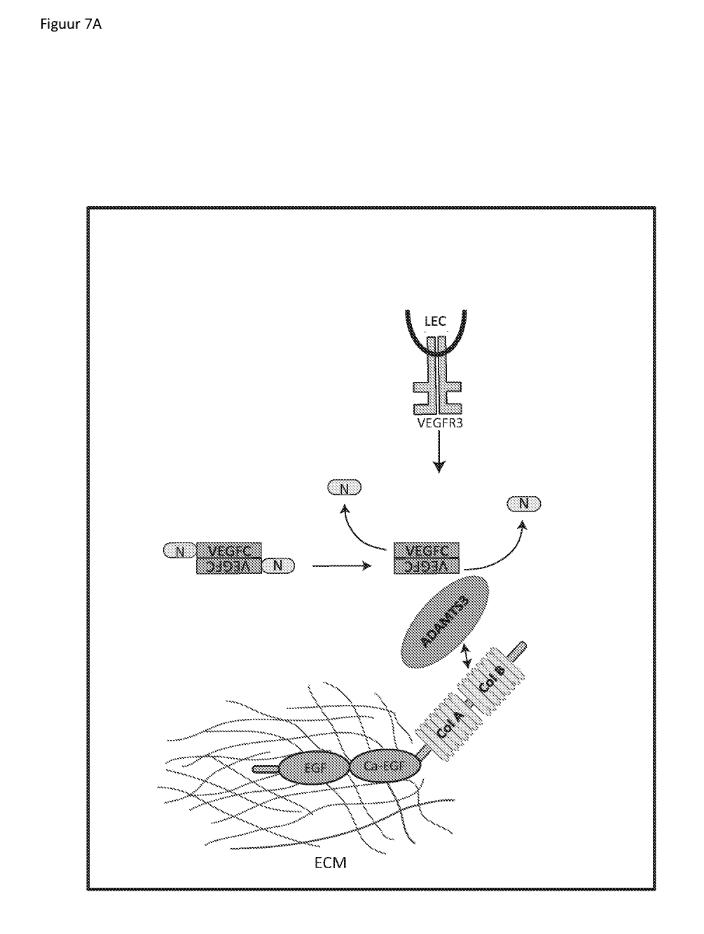

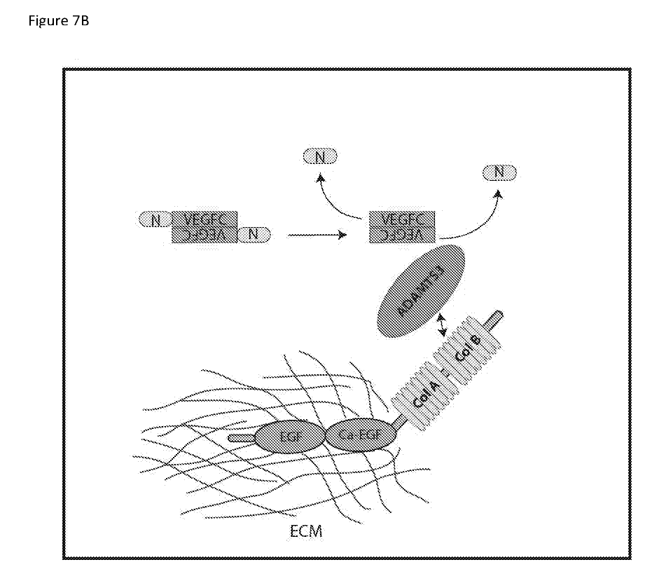

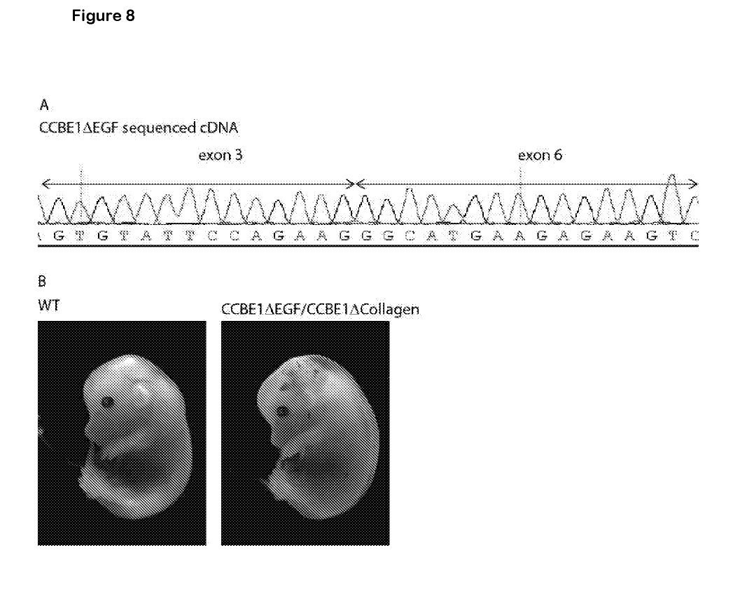

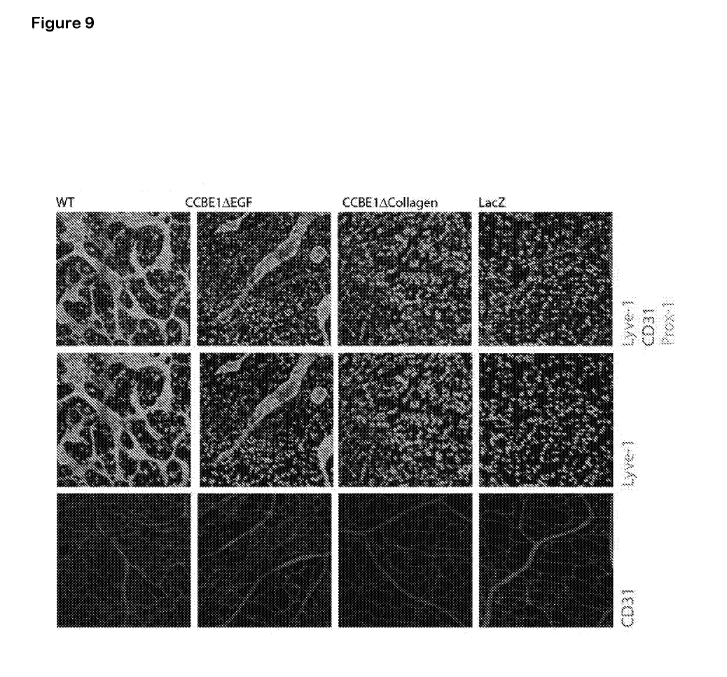

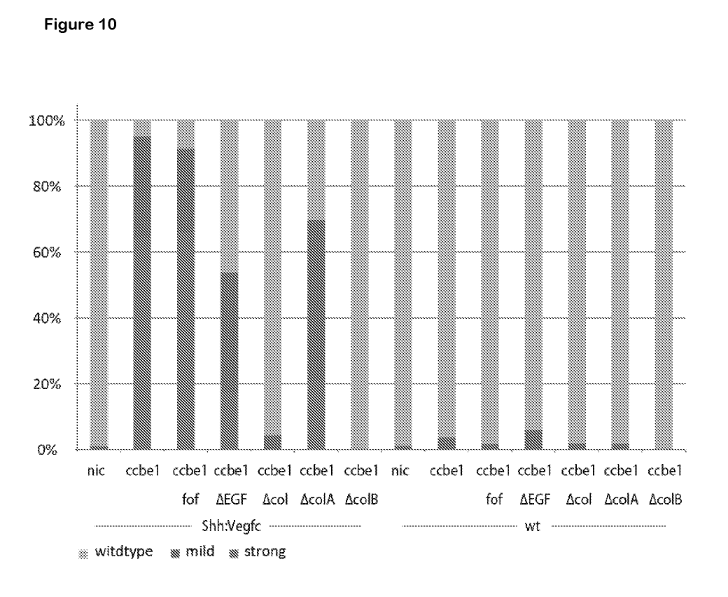

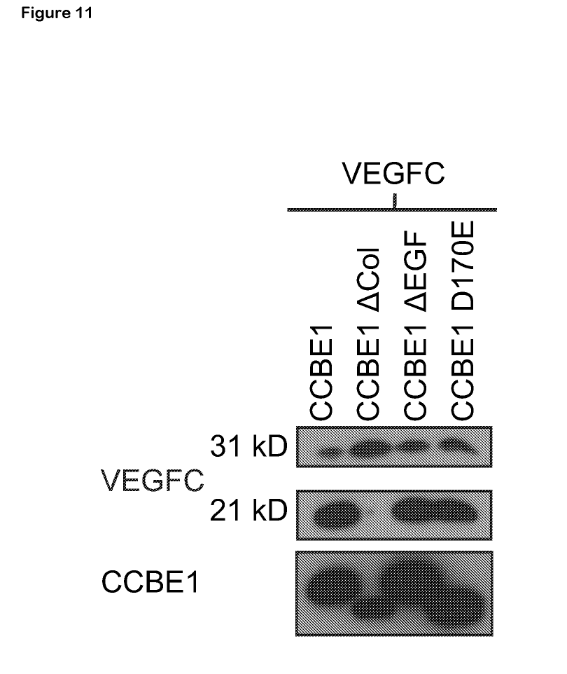

To investigate the relevance of the different domains of CCBE1 in vivo, we generated knock-in mice expressing different deletion mutants of CCBE1, lacking either the EGF and Ca-EGF domains (CCBE1ΔEGF mice) or lacking both collagen repeat domains (CCBE1ΔCol mice) (FIG. 1 A). In order to remove the EGF domains in CCBE1, we generated mice with floxed exons 4 and 5 (FIG. 1 B). Cre-mediated recombination led to an in-frame deletion of exon 4 and 5 removing both the EGF domains. Correct splicing from exon 3 to 6 was verified by sequencing of cDNA from homozygous CCBE1ΔEGF embryos (FIG. 8). For CCBE1ΔCol mice, removal of the two collagen repeat domains was achieved by replacement of exon 7 of CCBE1 with the cDNA of exon 7 to 11 lacking amino acids 248 to 337 followed by a transcriptional stop (FIG. 1 C). Analogous to this knock-in, a CCBE1 full length (FL) knock-in mouse was generated as a control in which exon 7 is replaced with the cDNA of exon 7 to 11 including the collagen domains followed by a transcriptional stop. Homozygous CCBE1FL control knock-in mice were viable and fertile and did not show any overt morphological defects (FIG. 1D). Homozygous fetuses expressing CCBE1ΔEGF or CCBE1ΔCol present with severe edema at E14.5 and die in utero, thus phenocopying the CCBE1 full knock-out in which a LacZ cassette was placed into the CCBE1 locus (CCBE1LacZ)==2 (FIG. 1D). Heterozygous animals for both CCBE1ΔEGF and CCBE1ΔCol mutants were viable and fertile and did not show any overt edemic phenotype, but fetuses compound heterozygous for CCBE1ΔEGF and CCBE1ΔCol show the same phenotype as mice homozygous for the two alleles (FIG. 8).

Thus, mutant CCBE1 proteins lacking either the EGF domains or the collagen repeat domains cannot substitute for wild type CCBE1 in vivo.

CCBE1ΔCollagen Mice Lack all Lymphatic Structures, but Lumenized Lymphatic Structures Develop in CCBE1ΔEGF Fetuses.

Previously, we have shown that CCBE1 knock-out mice lack all lymphatic vessels, while the patterning of blood vessels is not affected¹⁵. In order to analyze lymphatic and blood vessel development in mice expressing CCBE1 lacking either the EGF domains or the collagen repeat domains, we performed skin stainings of mutant fetuses at E14.5 to E16.5 (FIG. 2A, FIG. 9) using Lyve1 and Prox1 as lymphatic markers and the pan-endothelial marker CD31 to visualize blood vessels. We observed no abnormalities in blood vessels in any of the mice. Moreover CCBE1FL knock-in mice do not show obvious differences in lymphatic vessel development in the skin compared to wild type animals. Homozygous CCBE1ΔCol fetuses phenocopy CCBE1LacZ mice lacking all lymphatic vessels in the skin. Interestingly, however, CCBE1ΔEGF mice do exhibit some lymphatic structures in the skin. Like lymphatic vessels in wild type control fetuses, these structures sprouted into the direction of the dorsal midline at E14.5, but they failed to develop into a contiguous, branched plexus compared to wt control fetuses at both time points analyzed. At E16.5, patches of lymphatic endothelial cells had aggregated and formed large circular structures. Next we performed Lyve-1 stainings of paraffin sections of E14.5 fetuses (FIG. 2B) to investigate lymphatic development at other positions close to the CV and to assess whether these vessels are lumenized. While the pTD and other large lymphatic vessels in other areas such as the skin could be readily identified in wild type control and in CCBE1FL knock-in fetuses, lymphatic vessels were undetectable in CCBE1ΔCol or CCBE1LacZ mice. Analogous to the whole mount stainings, lymphatic vessels were found in the skin of E14.5 CCBE1ΔEGF fetuses. Moreover, lymphatic structures were also identified at the position of the pTD; however, these structures were smaller than in wild type control animals.

Collectively, these results reveal that while both the collagen domains and the EGF domains are necessary for lymphatic development, a CCBE1ΔEGF mutant does retain partial functionality in vivo.

Specified LECs Migrate from the Cardinal Vein in CCBE1ΔEGF Embryos, but not in CCBE1ΔCollagen or CCBE1LacZ Embryos.

In order to analyze lymphatic development in more detail and at an earlier stage, we visualized the formation of the first lymphatic structures by whole mount staining and optical clearing of E12.5 embryos. Recently, it had been shown that specified LECs leave the CV as streaks of cells to form the first two large, lumenized lymphatics vessels, the PLLV and the pTD. In CCBE1LacZ knock-out embryos as well as in CCBE1ΔCol embryos, Prox1+ or Lyve1+ LECs are unable to leave the CV and were not detected outside of the CV. In CCBE1ΔEGF mice however, some Prox1+ cells were able to leave the cardinal vein but only formed discontinuous lymphatic structures at the positions where the pTD and PLLV are normally located (FIG. 3).

Thus, the CCBE1ΔCol mutant has no activity in vivo. The CCBE1ΔEGF mutant is able to induce migration of lymphatic progenitor cells out of the CV at early time points of lymphatic development, although it cannot induce the formation of the main lymphatic structures, the PLLV and pTD.

The Second Collagen Domain of Ccbe1 is Essential for Enhancing Vegfc Signaling in Zebrafish.

To confirm and extend our findings in an independent system, we employed an assay that assesses Vegfc signaling in zebrafish I-U. Previously we have shown that Tg(shh:regfc;shh:ccbe1) zebrafish, expressing both regfc and ccbe1 as independent transgenes in the floorplate from the shh promoter, exhibit aberrant ectopic turning of arteries at 32 hpf¹⁰, while single transgenic zebrafish do not. This phenotype can be attributed to high Vegfc/Vegfr3 signaling in arteries². Injection of ccbe1 mRNA in Tg (shh:regfc) embryos that only express regfc from the shh promoter also efficiently induced strong arterial sprouting (FIG. 4B,H), while injection of ccbe1 mRNA into wt embryos had no effect (FIG. 10). First, we tested the activity of ccbe1fof in this assay. Ccbe1fof harbors a mutation in the EGF domain (D162E), leading to a loss of lymphatic development in zebrafish mutants¹². Unexpectedly, ccbe1fof mRNA has levels of activation that are indiscernible from wt ccbe1 (FIG. 4B, C, H). Interestingly and in accordance to the mouse data, ccbe1ΔEGF was still able to induce mis-turning of arteries, albeit to a lower degree than wt ccbe1 (FIG. 4D, H). In contrast, ccbe1ΔCol mRNA injection did not induce ectopic arterial turning (FIG. 4E,H). To further narrow down which part of the collagen domains was required for its function we tested mutant forms of ccbe1 in which either the first or the second collagen domain was removed. Interestingly, we found that deletion of the first collagen domain, ccbe1ΔColA, only led to a partial loss of function in the ability to induce ectopic turning whereas loss of the second collagen domain, ccbe1ΔColB led to a complete loss of function (FIG. 4 F-H).

Thus, the EGF and Ca-EGF domain as well as the first collagen domain are required for full function of the Ccbe1 protein, however, only removal of the second collagen domain fully renders the Ccbe1 protein dysfunctional in regulating Vegfc signaling in zebrafish. These data are in alignment with the above-described mouse mutant analysis.

The Second Collagen Domain of CCBE1 is Required for Enhancement of Proteolytic Processing of VEGFC by CCBE1 In Vitro.

CCBE1 has recently been shown to enhance proteolytic processing of VEGFC, which leads to an increase in bioavailability of the mature 21 KDa form of VEGFC^{10, 17}. To assess how the various functional domains affect the ability of CCBE1 to increase mature VEGFC levels we co-expressed human VEGFC together with wt or various mutant forms of CCBE1 in HEK293T cells. Conditioned media containing secreted VEGFC and CCBE1 were analyzed by Western blotting. As reported before, CCBE1 clearly stimulated the proteolytic processing of VEGFC as is apparent from an increased amount of the 21 KDa form of VEGFC. Surprisingly, we found that the EGF domains are not required for CCBE1 function in VEGFC processing in vitro as co-expression of CCBE1ΔEGF led to an increase in mature VEGFC comparable to that of wt CCBE1 (FIG. 5B). Moreover, CCBE1D170E, which harbors a point mutation that is equivalent to the fof mutation in zebrafish, was also able to enhance processing of VEGFC in this assay (FIG. 11), further corroborating that perturbation of the EGF domain does not impair CCBE1 mediated processing of VEGFC in vitro.

In contrast, the mutant form of CCBE1 that lacks the collagen domains (CCBE1ΔCol) lost the ability to increase VEGFC processing. As the zebrafish experiments indicated that the two collagen domains are not equally important for CCBE1 function in mediating VEGFC signaling in vivo, we tested whether these domains would also have differential effects on VEGFC processing in vitro. Strikingly, deletion of the first collagen domain (CCBE1ΔColA) was dispensable for activation of VEGFC processing in this assay, however, deletion of the second collagen domain (CCBE1ΔColB) inhibited activation of VEGFC processing by CCBE1 (FIG. 5C). To exclude a role for the amino acids that lie between the two collagen domains we deleted amino acids 291-299 and showed that this alteration does not affect CCBE1 function. Moreover, even deletion of amino acids 92 to 294, which deletes all parts of the protein from the EGF domains to the first collagen domain, did not have an overt effect on CCBE1 function in this assay (FIG. 5D).

Since CCBE1 has been suggested to mediate processing of VEGFC via the ADAMTS3 protease¹⁷, we also tested if ADAMTS3 cooperates with mutant forms of CCBE1. We generated a stable cell line expressing VEGFC and subsequently co-expressed ADAMTS3 and CCBE1. We found that ADAMTS3 on its own induced a mild increase of proteolytic processing which was further increased by co-expression of CCBE1 and CCBE1ΔEGF. This induced a strong loss of the 31 kD form and a concomitant increase of 21 kD form of VEGFC which was never seen in the absence of ADAMTS3. In agreement with our other findings, lack of the collagen domains abrogates the synergism between CCBE1 and ADAMTS3 (FIG. 5E).

Taken together, these data suggest that the collagen domains are indispensable for processing of VEGFC by CCBE1, however, all other functional domains tested are dispensable for its activity in vitro.

Only a Hennekam Mutation in the Collagen Domain, but not in the Ca-EGF Domain Fully Impairs CCBE1 Function.

CCBE1 mutations have been shown to be causative for HS, a congenital autosomal recessive disease, which is characterized by lymphangiectasia, generalized lymphoedema and mental retardation¹⁸. Most of the mutations inducing HS have been identified affecting the N-terminal part of the protein, containing a cysteine rich sequence N-terminal of the EGF domains and the EGF and Ca-EGF domains (FIG. 6A). Only two mutations have been found within the collagen domains, with one patient harboring a mutation in the second collagen domain, G327R. To interrogate the effect of the mutations responsible for the HS phenotype on the functionality of CCBE1, we tested two constructs that bear mutations found in HS patients in our assays. One mutation, R158C, is located within the Ca-EGF domain and one mutation, G327R, within the second collagen repeat. First, we tested the functionality of these two mutants in vivo by injecting zebrafish ccbe1 mRNAs that bear the corresponding mutations into Tg(shh:vegfc) embryos. Whereas the zebrafish R150C mutation, equivalent to human R158C and located in the Ca-EGF domain, only marginally lowered activity compared to wt ccbe1 in this assay, the mutation in the collagen domain, G313R in zebrafish, completely abolished Ccbe1 function (FIG. 6B,C). Moreover, in an in vitro processing assay using human CCBE1 the R158C mutation in the Ca-EGF domain did not affect functionality of CCBE1 compared to the wild type molecule, while the G327R mutation in the second collagen domain led to a strong loss of VEGFC activation (FIG. 6D).

Discussion

CCBE1 is one of the very few genes that are indispensable for embryonic lymphatic development¹. It has been shown that CCBE1 is a crucial regulator of VEGFC activation via the protease ADAMTS3. However, which domains of CCBE1 are necessary to exert its function is not known. Here we have conducted a functional domain analysis, based on different in vitro and in vivo systems, to gain comprehensive insight into the requirement of the different conserved protein domains for CCBE1 activity.

Several lines of evidence have stressed the importance of the EGF domains in the past. First, the majority of mutations in CCBE1 in HS patients have been found within, or in close proximity to the EGF domains. Second, in the original screens that identified zebrafish ccbe1 as an essential gene for lymphatic development, several alleles were identified in which the causative mutation was found to be located in the EGF or Ca-EGF domain^{12, unpublished data}. To gain more insight into the function of the EGF and Ca-EGF domains, we generated knock-in mice expressing a CCBE1 variant lacking these domains. Surprisingly, unlike the full CCBE1 knock-out fetuses which lack all lymphatic structures, CCBE1ΔEGF fetuses do develop rudimentary, lumenized lymphatic vessels in the skin and at the position of the pTD and PLLV. In the skin, these lumenized larger structures sprouted into the same direction to the dorsal midline as lymphatic vessels in wt fetuses. They did, however, fail to form a properly branched lymphatic network. In CCBE1ΔEGF embryos, remnant, fragmented lymphatic structures were located at the position of the pTD and PLLV indicating that specified LECs do migrate out of the cardinal vein roof to the same location as LECs in wild type control embryos. In accordance with this, a Ccbe1ΔEGF mutant was partially functional in zebrafish, were the injection of ccbe1ΔEGF can still exert aberrant arterial sprouting in the Tg(shh:vegfc) zebrafish. Although ccbe1fof could increase Vegfc signaling in arteries to the same extent as wt ccbe1, it cannot substitute for wt ccbe1 in zebrafish since, in contrast to wt ccbe1 mRNA, it is not able to rescue a ccbe1 mutant¹². Unexpectedly, loss of the EGF domains did not have any effect on the ability of CCBE1 to enhance processing of VEGFC in tissue culture cells. Thus, in vitro, the EGF and Ca-EGF domains are dispensable for the function of CCBE1 in VEGFC activation, and we would assume that this also holds true in vivo. However, as a CCBE1ΔEGF molecule cannot substitute for wt CCBE1 in mice and has reduced activity in increasing Vegfc signaling in zebrafish, the EGF domains do have a crucial function in vivo. The precise role of the EGF and Ca-EGF domains in vivo is not yet clear and needs further investigation in the future. It might be that this domain is necessary in other processes such as binding to ECM components. Consistent with this notion, a CCBE1ΔCollagen-Fc protein has been shown to bind to vitronectin, Collagen I, Collagen IV and Collagen V in an in vitro binding assay¹⁵. Alternatively the EGF domains could be involved in other aspects of VEGFC/VEGFR3 signaling such as receptor binding, possibly also mediated through prior or simultaneous interaction of CCBE1 with ECM components. Indeed, previously it has been shown that a recombinant protein that only contains amino acids 1-175 of CCBE1 was able to increase VEGFR3 phosphorylation when recombinant CCBE1 and recombinant pro-VEGFC were added to a culture of porcine aortic endothelial cells¹⁷. In this assay, CCBE1 increased binding of both pro-VEGFC and mature VEGFC to the receptor. Thus, it is tempting to speculate that the EGF domains are required to enhance binding of VEGFC to VEGFR3. However, we cannot exclude that if provided in high amounts such as through exposure as a purified recombinant protein, also the N-terminal part of CCBE1 can induce VEGFC processing. In a different assay, a cornea pocket assay, the N-terminal part of CCBE1 was able to enhance the lymphangiogenic effect of purified, fully processed VEGFC also indicating that the EGF domains of CCBE1 might be involved in other aspects of VEGFC signaling, perhaps enhancing binding of VEGFC to VEGFR3.

In all three different model systems tested we show that loss of the collagen domains has a profound effect on CCBE1 function. CCBE1ΔCol knock-in mice phenocopy the CCBE1 LacZ full knockout mice characterized by a failure of specified lymphatic endothelial cells to leave the cardinal vein. This was further confirmed by a total lack of enhancement of VEGFC signaling by ccbe1ΔCol in Tg(shh:vegfc) zebrafish and the inability of CCBE1ΔCol to induce proteolytic processing of VEGFC. In particular, the second collagen domain was found to be of higher importance, as both our zebrafish and our in vitro experiments show that loss of the second collagen domain alone strongly reduce the function of the CCBE1 protein. The crucial role of the collagen domains may be due to their involvement in direct activation of ADAMTS3. This is supported by the fact that co-expression of ADAMTS3 and CCBE1ΔCol did not synergize in VEGFC processing.

Our findings shed new light on the molecular defects that underlie HS and may provide a rationale that explains why so few HS mutations were found in the collagen domains. To date, most patients exhibit missense mutations affecting the N-terminal domain clustered in the EGF domain, the Ca-binding EGF domain or the cysteine rich domain upstream of the EGF domain. We could show that a mutation within the Ca-EGF domain inducing HS, C158R, and a mutation that leads to lymphatic defects in zebrafish, Ccbe1fof, does not affect the ability of CCBE1 to activate VEGFC processing in vitro and can still enhance Vegfc signaling in vivo, indicating that these mutations are hypomorphic alleles. In fact, the phenotype of ccbe1fof mutant zebrafish can partially be rescued by expressing high levels of full length Vegfc in the floorplate of zebrafish E. These results and the fact that a CCBE1ΔEGF molecule is partially active in vivo suggest that the HS mutations found within the EGF and EGF-Ca domains of CCBE1 are most likely hypomorphic alleles that retain some function.

In contrast, only one out of 13 patients identified so far harbors a mutation within the second collagen domain. This mutation, G327R, probably affects the secondary structure of the collagen repeat¹⁹. In our analyses we found that the G327R mutation strongly reduced CCBE1 function in vitro and in vivo, almost completely impairing its ability to activate VEGFC processing. This result is surprising as such a strong loss of function would most likely result in a complete absence of lymphatics and it is unlikely that many patients could survive that have mutations in the collagen-B domain. Therefore we consider it more likely that predominantly weaker alleles of CCBE1 are viable and can be found in patients, e.g. alleles that affect the EGF domain. Full loss-of-function situations are unlikely to be viable in humans. Of note, the only known patient bearing the G327R mutation is the only surviving child out of four siblings¹³. Furthermore, this patient is the only patient known to exhibit vascular defects in addition to lymphatic defects indicating that this mutation has a stronger effect than the other HS mutations found¹⁹.

Taken together our findings show that the collagen domains of CCBE1 are indispensable for its function in VEGFC activation in vitro and in vivo. The EGF domains are dispensable for this function in vitro, but are necessary for full activity of Ccbe1 in enhancing Vegfc/Vegfr3 signaling in vivo and for proper lymphangiogenesis in the mouse embryo. Contrary to the intuitive belief that the collagen domains bind to the ECM and that the EGF domain is more functionally relevant, we show here that it is the collagen repeat domain that is absolutely essential for enhancement of proteolytic processing of VEGFC by CCBE1, presumably by activating ADAMTS3. The role of the EGF domain should be subject of future investigations but previous work has indicated their capacity to bind ECM proteins. In zebrafish, ccbe1 is expressed along the migration route of lymphatic precursor cells; thus, it is possible that binding of the EGF domains to ECM components serves to direct specific locations of CCBE1 activity. At these dedicated areas the collagen repeat domains of CCBE1 mediate enhanced processing of VEGFC by activation of ADAMTS3. Thus, CCBE1 provides positional information for VEGFC signaling, which orchestrates the migration of LECs (FIGS. 7A and 7B). These molecular insights provide new entry points for therapeutical approaches to either stimulate or inhibit lymphangiogenesis by modulating CCBE1 function.

### CITED ART

1. Schulte-Merker S, Sabine A, Petrova T V. Lymphatic vascular morphogenesis in development, physiology, and disease. J Cell Biol. 2011; 193:607-618
2. Alitalo K. The lymphatic vasculature in disease. Nat Med. 2011; 17:1371-1380
3. Wigle J T, Harvey N, Detmar M, Lagutina I, Grosveld G, Gunn M D, Jackson D G, Oliver G. An essential role for prox1 in the induction of the lymphatic endothelial cell phenotype. EMBO J. 2002; 21:1505-1513
4. Yang Y, Garcia-Verdugo J M, Soriano-Navarro M, Srinivasan R S, Scallan J P, Singh M K, Epstein J A, Oliver G. Lymphatic endothelial progenitors bud from the cardinal vein and intersomitic vessels in mammalian embryos. Blood. 2012; 120:2340-2348
5. Hagerling R, Pollmann C, Andreas M, Schmidt C, Nurmi H, Adams R H, Alitalo K, Andresen V, Schulte-Merker S, Kiefer F. A novel multistep mechanism for initial lymphangiogenesis in mouse embryos based on ultramicroscopy. EMBO J. 2013; 32:629-644
6. Kuchler A M, Gjini E, Peterson-Maduro J, Cancilla B, Wolburg H, Schulte-Merker S. Development of the zebrafish lymphatic system requires vegfc signaling. Curr Biol. 2006; 16:1244-1248
7. Yaniv K, Isogai S, Castranova D, Dye L, Hitomi J, Weinstein B M. Live imaging of lymphatic development in the zebrafish. Nat Med. 2006; 12:711-716
8. van Impel A, Schulte-Merker S. A fisheye view on lymphangiogenesis. Advances in anatomy, embryology, and cell biology. 2014; 214:153-165
9. Hogan B M, Herpers R, Witte M, Helotera H, Alitalo K, Duckers H J, Schulte-Merker S. Vegfc/flt4 signalling is suppressed by dll4 in developing zebrafish intersegmental arteries. Development. 2009; 136:4001-4009
10. Le Guen L, Karpanen T, Schulte D, Harris N C, Koltowska K, Roukens G, Bower N I, van Impel A, Stacker S A, Achen M G, Schulte-Merker S, Hogan B M. Ccbe1 regulates vegfc-mediated induction of vegfr3 signaling during embryonic lymphangiogenesis. Development. 2014; 141:1239-1249
11. Karkkainen M J, Haiko P, Sainio K, Partanen J, Taipale J, Petrova T V, Jeltsch M, Jackson D G, Talikka M, Rauvala H, Betsholtz C, Alitalo K. Vascular endothelial growth factor c is required for sprouting of the first lymphatic vessels from embryonic veins. Nature immunology. 2004; 5:74-80
12. Hogan B M, Bos F L, Bussmann J, Witte M, Chi N C, Duckers H J, Schulte-Merker S. Ccbe1 is required for embryonic lymphangiogenesis and venous sprouting. Nat Genet. 2009; 41:396-398
13. Gordon K, Schulte D, Brice G, Simpson M A, Roukens M G, van Impel A, Connell F, Kalidas K, Jeffery S, Mortimer P S, Mansour S, Schulte-Merker S, Ostergaard P. Mutation in vascular endothelial growth factor-c, a ligand for vascular endothelial growth factor receptor-3, is associated with autosomal dominant milroy-like primary lymphedema. Circ Res. 2013; 112:956-960
14. Gordon K, Spiden S L, Connell F C, Brice G, Cottrell S, Short J, Taylor R, Jeffery S, Mortimer P S, Mansour S, Ostergaard P. Flt4/vegfr3 and milroy disease: Novel mutations, a review of published variants and database update. Hum Mutat. 2013; 34:23-31
15. Bos F L, Caunt M, Peterson-Maduro J, Planas-Paz L, Kowalski J, Karpanen T, van Impel A, Tong R, Ernst J A, Korving J, van Es J H, Lammert E, Duckers H J, Schulte-Merker S. Ccbe1 is essential for mammalian lymphatic vascular development and enhances the lymphangiogenic effect of vascular endothelial growth factor-c in vivo. Circ Res. 2011
16. Joukov V, Sorsa T, Kumar V, Jeltsch M, Claesson-Welsh L, Cao Y, Saksela O, Kalkkinen N, Alitalo K. Proteolytic processing regulates receptor specificity and activity of vegf-c. EMBO J. 1997; 16:3898-3911
17. Jeltsch M, Jha S K, Tvorogov D, Anisimov A, Leppanen V M, Holopainen T, Kivela R, Ortega S, Karpanen T, Alitalo K. Ccbe1 enhances lymphangiogenesis via a disintegrin and metalloprotease with thrombospondin motifs-3-mediated vascular endothelial growth factor-c activation. Circulation. 2014; 129:1962-1971
18. Alders M, Hogan B M, Gjini E, Salehi F, Al-Gazali L, Hennekam E A, Holmberg E E, Mannens M M, Mulder M F, Offerhaus G J, Prescott T E, Schroor E J, Verheij J B, Witte M, Zwijnenburg P J, Vikkula M, Schulte-Merker S, Hennekam R C. Mutations in ccbe1 cause generalized lymph vessel dysplasia in humans. Nat Genet. 2009; 41:1272-1274
19. Alders M, Mendola A, Ades L, Al Gazali L, Bellini C, Dallapiccola B, Edery P, Frank U, Hornshuh F, Huisman S A, Jagadeesh S, Kayserili H, Keng W T, Lev D, Prada C E, Sampson J R, Schmidtke J, Shashi V, van Bever Y, Van der Aa N, Verhagen J M, Verheij J B, Vikkula M, Hennekam R C. Evaluation of clinical manifestations in patients with severe lymphedema with and without ccbe1 mutations. Molecular syndromology. 2013; 4:107-113
20. Lawson N D, Weinstein B M. In vivo imaging of embryonic vascular development using transgenic zebrafish. Dev Biol. 2002; 248:307-318
21. Bohmer R, Neuhaus B, Buhren S, Zhang D, Stehling M, Bock B, Kiefer F. Regulation of developmental lymphangiogenesis by syk(+) leukocytes. Developmental cell. 2010; 18:437-449
22. Boisset J C, Clapes T, Van Der Linden R, Dzierzak E, Robin C. Integrin alphaiib (cd41) plays a role in the maintenance of hematopoietic stem cell activity in the mouse embryonic aorta. Biology open. 2013; 2:525-532

List of sequences

SEQ ID:NO 1
Human CCBE1 amino acid sequence, Accession code NM_133459
MVPPP PSRGG AARGQ LGRSL GPLLL LLALG HTWTY REEPE DGDRE ICSES KIATT
KYPCL KSSGE LTTCY RKKCC KGYKF VLGQC IPEDY DVCAE APCEQ QCTDN FGRVL
CTCYP GYRYD RERHR KREKP YCLDI DECAS SNGTL CAHIC INTLG SYRCE CREGY
IREDD GKTCT RGDKY PNDTG HEKSE NMVKA GTCCA TCKEF YQMKQ TVLQL KQKIA
LLPNN AADLG KYITG DKVLA SNTYL PGPPG LPGGQ GPPGS PGPKG SPGFP GMPGP
PGQPG PRGSM GPMGP SPDLS HIKQG RRGPV GPPGA PGRDG SKGER GAPGP RGSPG
PPGSF DFLLL MLADI RNDIT ELQEK VFGHR THSSA EEFPL PQEFP SYPEA MDLGS
GDDHP RRTET RDLRA PRDFY P
SEQ ID:NO 2
Human CCBE1 cDNA nucleotide sequence, Accession code NM_133459

| | |
|---|---|
| 1 | cggccgcgga ggagcaggac gcttggtccg gacggagctc ggcgctggga agaagccggg |
| 61 | agcttccctg atggtgccgc cgcctccgag ccggggagga gctgccaggg gccagctggg |
| 121 | caggagcctg ggtccgctgc tgctgctcct ggcgttggga cacacgtgga cctacagaga |
| 181 | ggagccggag gacggcgaca gagaaatctg ctcagagagc aaaatcgcga cgactaaata |
| 241 | cccgtgtctg aagtcttcag gcgagctcac cacatgctac aggaaaaagt gctgcaaagg |
| 301 | atataaattt gttcttggac aatgcatccc agaagattac gacgtttgtg ccgaggctcc |
| 361 | ctgtgaacag cagtgcacgg acaactttgg ccgagtgctg tgtacttgtt atccgggata |
| 421 | ccgatatgac cgggagagac accggaagcg ggagaagcca tactgtctgg atattgatga |
| 481 | gtgtgccagc agcaatggga cgctgtgtgc ccacatctgc atcaatacct tgggcagcta |
| 541 | ccgctgcgag tgccgggaag gctacatccg ggaagatgat gggaagacat gtaccagggg |
| 601 | agacaaatat cccaatgaca ctggccatga gaagtctgag aacatggtga aagccggaac |
| 661 | ttgctgtgcc acatgcaagg agttctacca gatgaagcag accgtgctgc agctgaagca |
| 721 | aaagattgct ctgctcccca acaatgcagc tgacctgggc aagtatatca ctggtgacaa |
| 781 | ggtgctggcc tcaaacacct accttccagg acctcctggc ctgcctgggg gccagggccc |
| 841 | tcccggctca ccaggaccaa agggaagccc aggcttcccc ggtatgccag gccctcctgg |
| 901 | gcagcccggc ccacggggct caatgggacc catgggacca tctcctgatc tgtcccacat |
| 961 | taagcaaggc cggaggggcc ctgtgggtcc accaggggca ccaggaagag atggttctaa |
| 1021 | gggggagaga ggagcgcctg ggcccagagg gtctccagga ccccctggtt ctttcgactt |
| 1081 | cctgctactt atgctggctg acatccgcaa tgacatcact gagctgcagg aaaaggtgtt |
| 1141 | cgggcaccgg actcactctt cagcagagga gttcccttta cctcaggaat ttcccagcta |
| 1201 | cccagaagcc atggacctgg gctctggaga tgaccatcca agaagaactg agacaagaga |
| 1261 | cttgagagcc cccagagact tctacccata gcacatccca acaccgtcac gccaaaggaa |
| 1321 | gagaaagatc aactcacctg cagttaaacc atctaaagag aagaaagacc actggagacc |
| 1381 | tagaaaacat acatttttct cttctcttct cctgacgtct ctccactcct cttcttccaa |
| 1441 | atacgatgct attttcagag tcccctccta ggcctgcaga catgagggag tgaatgattg |
| 1501 | atttacctgc ttctcactaa gagtccattg gggtggtttg cattgtaact tttcttttac |
| 1561 | atcctatttt tccaggaact ttggatttaa gtactctcac agtgtcttaa atcataaatt |
| 1621 | cttgaagtta aatttggcag agtatcaaaa gggggaaaat gacaaagtga gctctaagaa |
| 1681 | aatgtgaggc tacttctaag atgtgtgttc acaatagacc ataactcctc tagtatcaaa |
| 1741 | attggggctc ttcagttaaa aaggggtggg gaggacaaac gtgtcgatgt gctttggtgg |
| 1801 | agaatttttt ccttgtgctt ctagtagact ttaaatattg tatccctttg tcaaaccttg |
| 1861 | tttcccaaat tcaattaaag agaggagaga attgaatggc gtttagagaa gatagaaaag |
| 1921 | aatcacagtc atatatttac tgttatatag attgccacat tctaaaattc aaatacggtg |
| 1981 | cttaaggttt catgccatgc ttatctgtaa gtatcctatt tagggaagaa gattaaactc |
| 2041 | tcttttcaaa aaaacaaagt gaaatgcctg gattcacatt aaaacaatgg gctctcgttt |
| 2101 | gctataatat tttaaagctg tttaatcaac agtggagtct gctctataaa tatagattat |
| 2161 | ttgttcaata aactggctga gcttagagag aggtgcagaa ttcctggttc tgagcaggtg |
| 2221 | cccagaaggt accattaggt gccatgatcc aggctgaacc aatatacagt ggggctgaag |
| 2281 | tctgcaagga ggttgctggc ttgggctgac ctcactaatg ccatcagcag cggtaggtaa |
| 2341 | attttttctc cttgggtatt acaagttttt gtctggagcc aaccaagctt gccaccaaca |
| 2401 | tattgagagt aatacactat tgaaagttat cttggatggg gagaaaaaaa aatagtggtt |
| 2461 | ttccttgttt gcaaaaactt ccttcctatt ctcatttttt cttaattttc tttaatttag |
| 2521 | tccaagttcc agttctttta ggccttctct ttgatttatt ttcccctgca tgtgagaagc |
| 2581 | agttcagaaa aaggtctata tctccacctc ctagtgagtt agagtgtttt ctcagagcac |
| 2641 | ctctgggtgg caaagggaag catgttcctg ccaaggtttg ctgtggattc agaagcacca |
| 2701 | ggagcaagag accagaagga tgatctgctc ctttgtaacg ttgttgaggg ccctcttgtt |
| 2761 | tccaatgagc agcttatagg ttactcacag tccactttct cactggacac acaaagtggc |
| 2821 | tctttatcta cctttgcggg agattttcac tctcctgcaa atgatcgttc tcacactcat |
| 2881 | attagctcat gttggaattt cccatcctgc catgtccttt cccatttctt tttggctttt |
| 2941 | ttgcctccac cttttagccc acatcattta actccactac tgtgaaagct tgcttaaaga |
| 3001 | aaatccctct tggccgggtg tggtagccca cgcctctaat cccagcactt tgggaggctg |
| 3061 | aggcggggag atcacaaggt caggagatcg agaccagcct gaccaacatg gtgaaaccct |
| 3121 | gtctctacta aaaatacaaa aattagctgg gcgtgttggc acacacctgt aatcccagct |
| 3181 | actcaggagg ctgaggcagg agaattactt taacctgcgg ggggagccta gattgcgcta |
| 3241 | ctgcactcca gcctaggcaa cagagggaga ctctgtctca ttaaaaaaaa aaaaaaaaaa |
| 3301 | aaaaaaaggc cgggcttggt ggctcatgcc tgtaatccca gcactttggg aggccgaggc |
| 3361 | gggcggatca cgaggtcagg agatcgagac cacagtgaaa ccctgtctct actaaaaaca |
| 3421 | caaaaaatta gtcgggcgtg gtggtgggcg cctgtggtcc cagctgctcg ggaggctgag |
| 3481 | gcaggagaat ggcgtgaacc tgggaggcgg agcttgcgct gagccgagat cgtgccactg |
| 3541 | cactccagcc tgggcgacag agcgagactc tgtctcaaaa aaaaaaaaat atcccttttg |
| 3601 | ttgtgtttga atgccacctt tcagtcttct tccttaagaa tgttaggaag gggctttgca |
| 3661 | aacctctagt ggcaacagtc ttccttccct tcttcctacc acgttggaaa tgcaggactc |
| 3721 | atgtcagtca gcatgtgacc gagtctttca tgaacacttt gctcctctat gtctcattta |
| 3781 | tgcaaaattc ctggtgactc tccactaaca tttttaaagt gaaagcaaac tttcacttta |
| 3841 | aaaaaagtga gcgagaggat cccttgagcc caggagttca aggccagtct gggcaacata |
| 3901 | gcaagacccc atccctaaaa aaaaaaaaat aagttttatt aaatgtggca ctttccccta |
| 3961 | gaagggcagt aaccccagga accagtctgc ctcttttcga ataaaggaga ctttttaaaa |
| 4021 | agaaaaaatt tctttttagg gtggggtgct ctttatctag catctttcct tcctccttct |
| 4081 | ctctgcaaaa ttcaacattc atgacagagg gccacaccaa ccacgggttt atagagagag |
| 4141 | gaactgaaca gctgttgatt cccatctgga atatatttaa tgtttgcctg cttgtggagc |
| 4201 | atggtattta aagggtgtgg atggatgaat gtttaaaaaa aaacacacaa aactatgcac |
| 4261 | aagctgtatg agagatggct aggtccatct atgatttttt ttaaaagaag aattttaata |
| 4321 | ccttttcttt gacagtgaac tgtatttaga aatatgatct ttattgtgaa atgctcataa |
| 4381 | aatgcaacca aactgcatgt ttggccatag gctagaataa tatattaaaa gaattaatgg |
| 4441 | aaagtagtat tgtctgggtt tcatgaaact attccagata atattctcca gcaaaactta |
| 4501 | tggtaacctg tattttataa tgtcaatctg ctggaccaag ttaatttaga aaataatttt |
| 4561 | taatcctcag attttcaaaa cttggactac tgcaagactg atatgtactt tttatgccca |
| 4621 | aatcatattt ttcaaagata ggacatcata aggcatcatt atttctaatg tatcttttaa |
| 4681 | ctagatagaa aaagctttac tgtctaaaca ggacttctgc tcaaaaccta atgggaaagc |
| 4741 | tatttctggg gtaagatttg taacagtgct tctaggtcat ttcatgtttt atagggaaaa |
| 4801 | agatctttgt acacctcctc aacctgttag tagaaatgtt gacttcgatt ttttgcaccc |
| 4861 | tcttcacagg acctcataag ttgcctctgt tcttggaaag caatgggaat aaaaatatgc |
| 4921 | tgttgaaaat agtgcatggg gccaggcaca gtggttatgc ctgtaatccc aacactttgg |
| 4981 | gaggctgagg cgggcgcatc atgaggtcag gagatcaaga ccatcctgac caacacgtgg |
| 5041 | tgaaacccca tctctactaa aataaataaa aaaaaaaatt agccaggcgt ggtggtgcac |
| 5101 | gcctgtagtc tcagctactc gggaagctga gacacaagaa ttgcttgaac ccgggaggtg |
| 5161 | gaggttgcag tgagccaaga ttgcgccact gcactccagc ctggctacag agtgagactc |
| 5221 | cgtctcgaaa aaagaaaata gtacatggag ggcatgcatt ttagtttagg aaacccccct |
| 5281 | cccttcccag ttccagaggg agaaattata agctatactg tgccaaaagt gagtggggct |
| 5341 | gcagagagaa tggtgccttt tctcaccttc tgagttaatg cccgacatct gccattggat |
| 5401 | tcattgacca aaggaagtca tttctactgt ataaactcag gagtatcata gcccaacaag |
| 5461 | taaataataa atgttcattg gttttggaag gaaggaaagg tgagtattac cccaccaaat |
| 5521 | accagcgaag agcaccctgg ctttggccat ctttccaagg ttcataattg ctgtgggcct |
| 5581 | ggtgagccct gttcccaggt atgcaaggga gggcttctcc aggccagaca tttctgtagg |
| 5641 | ttttcaggta gcaactcccc catcatattt tgggtgtcat tccccactct ctctccccac |
| 5701 | ttctcagcct gtgccccaga aacaaccaca gcagcctcat tctgcagact taacatctgt |
| 5761 | caaataacct tcctgaaaag gaaagttatt ggaggaatcc aggccaaaca acaagtagaa |
| 5821 | tagagccatc taaagttatt aataatgagt tcagggaaat agatgaattt tttcctggca |
| 5881 | tagtcaatat ttatatctca caacaagtca aaaataattt tccatttgga aaacaaaaca |
| 5941 | caccatgact gtaattgtta aggcatataa tcatgaactt cacatgctcc ctcaacaaac |
| 6001 | gttactgctc tagggaaatc atagaaaagg accctcttac tccccagggg atcaaagcaa |
| 6061 | gtccccttag gaatttcatg tgtgccgtgg gtctgttttt atttcacagt gactagtcat |
| 6121 | atactggagt agctctgttg ttcattattt gtaacaggtc catgtaacag tcaagttagc |
| 6181 | attcaagcaa ttatggatgt gatactatga tgtacttttg ttatgattgt atatgcttaa |
| 6241 | taacaaagtt atttttctta aaaaaaaaaa aaaaaa |

SEQ ID: NO 3
Mus musculus CCBE1 amino acid sequence, Accession code NM_178793.4
MVPPP LPSRG GAAKR QLGKS LGPLL LLLAL GHTWT YREEP EDRDR EVCSE NKITT
TKYPC LKSSG ELTTC FRKKC CKGYK FVLGQ CIPED YDICA QAPCE QQCTD NFGRV
LCTCY PGYRY DRERH QKRER PYCLD IDECA TSNTT LCAHI CINTM GSYHC ECREG
YILED DGRTC TRGDK YPNDT GHEEK SENEV KAGTC CATCK EFSQM KQTVL QLKQK
MALLP NNAAE LGKYV NGDKV LASNA YLPGP PGLPG GQGPP GSPGP KGSPG FPGMP
GPPGQ PGPRG SMGPM GPSPD LSHIK QGRRG PVGPP GAPGR HGSKG ERGAP GPPGS
PGPPG SFDFL LLVLA DIRND IAELQ EKVFG HRTHS SAEDF PLPQE FSSYP ETLDF
GSGDD YSRRT EARDP EAPRN FYP
SEQ ID: NO 4
Mus musculus CCBE1 cDNA nucleotide sequence, Accession code
NM_178793.4

| | |
|---|---|
| 1 | gaaggcggct gcggccgcgg agcctccctg ggcacagcgc agggagagag ctcgggcgcc |
| 61 | gagaagaagc cgcgagcttc cccgatggtg ccgccgcctc ttccgagccg gggaggagca |
| 121 | gccaagaggc agctgggcaa gagcctgggg ccgctgctgc tactcctggc gctgggacac |
| 181 | acgtggacct accgagagga acccgaggac cgcgacagag aagtgtgctc tgaaaacaag |
| 241 | atcaccacga ccaaataccc atgtctgaag tcttcgggcg agctcaccac gtgcttcagg |
| 301 | aaaaaatgct gcaaaggata taaatttgtt cttggacagt gtattccaga agattatgac |
| 361 | atttgtgccc aagctccctg tgaacagcag tgcacggata acttcggccg tgtgctgtgc |
| 421 | acgtgctacc cgggataccg atatgaccgc gagagacacc aaaagcggga gagaccgtac |
| 481 | tgtctggata tcgatgaatg tgccaccagc aataccactc tctgtgcaca tatctgcatc |
| 541 | aacaccatgg gcagctacca ctgtgagtgt cgggagggct acatccttga ggacgatggg |
| 601 | aggacctgca caaggggaga caaatacccc aatgacactg ggcatgaaga gaagtccgag |
| 661 | aatgaggtga aagccgggac ctgctgcgcc acatgcaaag agttctccca gatgaagcag |
| 721 | accgtgctgc agctgaaaca gaagatggcg ctgctcccca acaatgccgc tgaactgggc |
| 781 | aagtatgtca atggtgacaa ggtactggcc tcaaacgcct accttccagg acctcctggc |
| 841 | ctgcctgggg gacagggccc accaggctca ccaggaccca aggggagccc tggattcccc |
| 901 | ggtatgccag gccctcctgg acagccaggc ccacggggct caatgggacc tatgggacct |
| 961 | tcccctgacc tgtctcatat taagcaaggc cggaggggcc ccgtgggccc accaggagca |
| 1021 | ccaggaagac acggctccaa gggggagaga ggagcacccg gacccccggg gtctccggga |
| 1081 | cccccaggtt ctttcgactt cctgctgctt gtgctggcgg acattcgaaa tgacatcgca |
| 1141 | gagctgcagg agaaggtgtt tggacaccgg actcactctt ccgccgagga cttcccccta |
| 1201 | cctcaggaat tctccagcta cccagagacg ctggactttg gctctggaga tgactactcc |
| 1261 | agaagaactg aagcaaggga ccccgaggcc ccaagaaatt tttatccata gcacatccca |
| 1321 | tatcctcacg ccaccagaag agaagcatgc cttcgacctt ccccttactc tgtttccaaa |
| 1381 | cccggttttc caagtttcct gtgcatcaca ctagcaacag gaagatgtga tgggttaacg |
| 1441 | tgctgttctt gaagagccaa tgggtggttg acatcaagac tttcttcaac gtccgatttt |
| 1501 | ttcaagagct tttgggtttc tgagttcatc attccacaga atcttcaact ttggcagagt |
| 1561 | gcaaaagagg ggaaaatggc accgtgggtt cttaggagtg taaggttcct tctaaaaaga |
| 1621 | aaataatgtt caaatgcttg actattcatt ccctactgtc aggagtaagg cttttagggt |
| 1681 | gggggtgggg gtggaggggc atggaaggag caaccatgga gaataacatt gatagaaaat |
| 1741 | gattttcctt agcaatatcc cagatttcaa attctgcatc ctattttcca acttgatggg |
| 1801 | ggtttttttt tgtttgtttg ttttggtttg gttttttgtt ttttgggggt gtttgtttgt |
| 1861 | ttgtttgtct ttaaatttgg ttacagaaac aagagattgg aaagaatgtg ttcagagaga |
| 1921 | tagagaatta gccttatatt tcctgtctca taggttgccg aatttagaat tcagatacga |
| 1981 | cagtcattga ttcgtgcaaa tcctatgtgg aggagggagt ttgtttgtaa gggcttgatc |
| 2041 | atgttttcta gggagcttgg ggtgaggaaa cactctgtgc atactagaac agagggctcc |
| 2101 | tggagaccag gacgattgaa gactacatgg tcaaaagcat tgtcctgctc catacatgtt |
| 2161 | gatgatttgt gaaataagat ggctgacatt taagaagaag tacaagatgc cctggcactg |
| 2221 | acagggttgc cagatgtgcc agcaaggcct gatccaggct gaatcaaggc tggagggaaa |
| 2281 | tcactgccct ggaatcttag ccaaggatgt caaggggaca ttgagaagtg cttcactaaa |
| 2341 | atcagattaa tcagaagcag attaatctac tcttaagaga tttgaagttc tcaccagggc |
| 2401 | acacttggaa cagcatacag ctgagaggtc tctgagatag agaaggagga ggagtttttg |
| 2461 | ataaattgga ataattttcc actatttgcc ccctgatttg gttagaatcc ctgttgttct |
| 2521 | gggtgtcctt gactttttgt cctgggccac aaggaaacgg cttggaggcc agcacatttc |
| 2581 | ccaatgtctc cctaagtccc ttcttggagt atgtctagat ccccagggaa tcatgttccc |
| 2641 | agaaaggagc aagaggccag caggttcctt cttggtatct ctgggttctt gtttcaggga |
| 2701 | tctgcttccg ggctccttag tgaacacttt ctccttgggt gtgcagcagg ctccgtgatc |
| 2761 | ttgctcacat ggttgcccac accagggtcc cagtttgcta tcctgcagtg cccccttata |
| 2821 | tttctaagaa gtttgagatt gcattgatct tttccctata tcacttaagc ctccatcgtc |
| 2881 | ttcccacacc tactgtgaca gcttgttgga ggaaacagct attggtttgc atgacactgc |
| 2941 | atccaccatc cactcttctt tctgagcaat aagaaggggc tctgtctaca gagccagcca |
| 3001 | gagcctcgtg ggctttcact tcccctctcc cacctcccta ccctcatccc tctctcctgg |
| 3061 | ttctcttcct tttccttttt tttctctctt cctctcctcc tcttttgctc cttctctcct |
| 3121 | tcatttcctc tacccctatc ctagcttccc tgcctttcca cctctccttt cttacttgct |
| 3181 | tccttcctcc ctccttccct ccctcccttt ctccttctct ccctccatct ttcttccttt |
| 3241 | ctactccctc cctcccttcc tacctaccta cttccttcct tccctttttt cttccttagt |
| 3301 | tccctctctc tgctccctcc ctccttccct ccatgcttgc ctgcctatct tcctacccag |
| 3361 | ttagaaaggc aggattccca cacactccca cccaaatccc cagtggctgt ccatcagcag |
| 3421 | tcctgatgaa ggcaagcaca cacttgcatc gcttgcttgc tgtctcaccc tccttccccc |
| 3481 | ttcagtctct cctgaacctg tgcatcacca cagatagcct tgtcaactgt ggcttcctga |
| 3541 | agagatggat gcaaccgcag tcaagtgcca cctggaatct atttagtgct tgcctgctta |
| 3601 | tagaacccat cttttaaagg gtgtggatgg atgaattctc aaaactatgc acaggctctg |
| 3661 | tgagcaataa ttatgtccat ctttttttat gaggaatttt ataactactt tttatgtcta |
| 3721 | ctttttttag acagtgaaat gtatgtagaa atataatctt tattgccaaa tgctggtaaa |
| 3781 | acgcgagcac actccatgct tggctggagg ccaggataat gtactgaaag aatgaagaga |
| 3841 | aagtcataat atctgggatt catgagtctg tctagataat tacctccagg aaaacataat |
| 3901 | agcctatatt ttgtgatctc aatatgccat gtcaatttag ataatcgtta ctcctcagat |
| 3961 | tttcaaaaca tggaatacta actaactaca agaccaacgc atgtcctcta cttattcaaa |
| 4021 | tcatggattt ttagtgataa agcattattg ctcccatatc ttttagttgt ggacagaaaa |
| 4081 | aaaaatcaaa gcttttctgt ctaaacaaat ttctgctcaa aatatcatag gaaggccttt |
| 4141 | gggaggaagg acagttcaga acaaatttac cgagtcactt catatctcac agggaaaata |
| 4201 | ttttagtttt ttatacgccc ttcaacactt tagtagaaat ggtcaacttt tctaatattt |
| 4261 | ccactggtta agacttggtt atgaattgcc tctgtgttta taaagcaatg agaataaaat |
| 4321 | ttcactgaaa ataatataaa gaagactttc attttagttt aggaaaactg aattttccaa |
| 4381 | ttccagaagg aaaaaaataa ccagttttcc tgtgccagtt aagtgttagg agctaggtgt |
| 4441 | gatggtggac acctgtaatc ccagcacctg agaagcacag ctaggcagat caggagttca |
| 4501 | aggtcagcct cagctacata acaagtgtga ggccagcctg ggctatctga gagtctatct |
| 4561 | caaaataata atgatgatga tgataatgtg atgataataa agaataataa ttagcagagg |
| 4621 | actaaagaga tggctcagag gttaaggatt cttgctgctc ttccagagat acagagctca |
| 4681 | gtttccagct cttgcacagg cagctcacaa catctgtaaa cacaggtcca ggtgatctgg |
| 4741 | cgccctcttc tggcttccat agtacctgca ctcacatgca cacacacaca tagatataca |
| 4801 | aacacataat tacaaataaa gtctttttta aaggagtggg tacaaagagg atgctgcctt |
| 4861 | ttcttgcttt ctatgttact ttccaatact tgctacggaa ttaactaaag tcatattgct |
| 4921 | gtgtggagcc aggccactat ccagcaggaa aattataagt gcccagtgat cttggaagaa |
| 4981 | gttttggccc atcaaaggcc aggtgcagag caccctggta gacccaggta agtcatgttc |
| 5041 | cctttccaag gttctgaaca tccttggcct tggaagtacc actatcaggc acattggcac |
| 5101 | aaaacttctt catgaccatc catcaaactt taccttgagg tctcaggaag ccacccccct |
| 5161 | atcttatttt gagcatcatt tttcattttc ctcctcttcc aaattcaaac agacagcctc |
| 5221 | aggagcctcc ttctgcaagc ttatcttcca gacaactgtt ctaaaaggga aagctattgg |
| 5281 | ctggaatctg agccatacaa ttagtaatat agaggcttct gttctattca gggaaataca |
| 5341 | tgggtcattt cctgatgcag caatcatgta tgtttcacaa catgtcaaaa ataattttct |
| 5401 | ctttggattt ttcaagggcc gtcattgtta aagctgaaat ggtgtgcttt atatactctt |
| 5461 | cctacaaatg ttaccatcag agaggaacca tgaaaaagga catatgggct atccatccct |
| 5521 | cagagagctc agtatgtttt gtccgattcc cagagcttga tccctgagtg gttcttgagt |
| 5581 | ctgtttttat ttctcagtca ctggtcatat atttcagtat ctctgtgcat tatttatatc |
| 5641 | agatcttgta acagtcaaac tgatattcca acagtcatgg atgtaatact aggaagtgcg |
| 5701 | tttgtatgac tgtatatgct taataacaaa gttatttttc tt |

SEQ ID: NO 5
Danio rerio CCBE1 amino acid sequence, Accession code NM_001163923
MIYPG RGASL SVAVA LVLFS SGAPW TFREE KEDVD REVCS ESKIA TTKYP CVKST
GEVTT CYRKK CCEGF KFVLG QCIPE DYDVC AGAPC EQQCT DHFGR VVCTC YDGYR
YDRER HRNRE KPYCL DIDEC ANNNE TVCSQ MCVNT PGSYR CDCHS GFYLE DDGKT
CTKGE RAPLF EKSDN VMKEG TCSAT CEDFH QMKMT VLQLK QKMSL LSSNT EINKQ
MTNEK MMMTT NSFLP GPPGP PGPAG TPGAK GSSGS PGQMG PPGLP GPRGD MGPIG
PSPDL SHIKQ GRRGP VGPPG APGRD GMKGE RGFPG PSGPP GPPGS FDFLL LMMAD
IRNDI AELQS KVFSR PLHSS FEDFP SAPDS WRDTP ENLDF GSGED YKSQS PPKSS
RKRKL PRNLK NPDWP V
SEQ ID: NO 6
Danio rerio CCBE1 partial cDNA nucleotide sequence, Accession code
NM_001163923

| | |
|---|---|
| 1 | ggaatctgcg cgtggggctt cgcaggaggt aaccaatcaa tactctccca attcctccga |
| 61 | aacaagcgct gaacttcaag actggattta aagggacaaa tatcttttaa tcatctcaga |
| 121 | gtgtctgaaa tgatctaccc gggcagagga gcatctctca gtgtggccgt ggcgctcgta |
| 181 | ctgttctcat cgggagctcc gtggactttc cgagaggaga aggaggatgt agacagagaa |
| 241 | gtgtgttcag aaagcaaaat cgcaacgacg aagtacccgt gcgtaaagtc cactggagag |
| 301 | gtgaccacat gctacagaaa gaagtgctgt gaaggtttca agtttgtcct gggtcagtgt |
| 361 | attccagaag attatgatgt gtgtgccggc gccccgtgtg aacagcagtg tacggatcat |
| 421 | ttcggtcggg ttgtttgcac ctgctatgac gggtatcgat atgaccggga acgacacaga |
| 481 | aacagggaga aaccatattg cctggacatt gatgaatgtg ccaacaataa cgagactgtg |
| 541 | tgctcacaga tgtgtgtgaa cactcccggc agctaccggt gcgactgcca cagcggcttc |
| 601 | tacctggagg atgatgggaa gacatgcacc aagggcgaga gagctccact ctttgaaaaa |
| 661 | tcagacaacg tcatgaaaga aggcacgtgt tcggccacat gcgaggactt ccatcagatg |
| 721 | aagatgactg ttctccaact caaacagaag atgtccttat tgtcgagcaa caccgagatc |
| 781 | aataagcaga tgaccaatga aaaaatgatg atgacgacaa attcattttt acctggacct |
| 841 | ccagggcctc ctgggcctgc agggacgcct ggagccaaag gatcctctgg ctctcctgga |
| 901 | cagatggggc caccaggctt acctggtccc agaggtgaca tgggacccat tggaccctcc |
| 961 | cctgacctgt cgcatatcaa acagggtcga cgtggcccag tgggtcctcc tggggctcca |
| 1021 | gggagagatg gcatgaaggg agagcgaggg tttcccggcc cttccggacc tccaggccca |
| 1081 | ccaggatcct ttgacttcct gttactcatg atggctgaca ttcgtaacga catcgctgag |
| 1141 | ctgcagagta aagtcttcag cagaccgtta cattcttcat ttgaggattt cccatctgcc |
| 1201 | ccggacagct ggagagacac accggagaac ctggacttcg gatccggcga agactacaaa |
| 1261 | tcccagagcc ctcccaagag cagcaggaag aggaaattac ctcggaactt aaaaaatccc |
| 1321 | gattggccgg tttgagactg tcagtaaaca atacaagaac tgaaaaaact gatggtgtaa |
| 1381 | atattacaga atgactgcta tttaactgta tactcgctga ggaaagaagg ctgaatatat |
| 1441 | ttcactgtat gaaatttcta tttttataac ataaaagatg tacatttttt gtaaaaaaaa |
| 1501 | aaaaaaaaaa aaaaaaa |

SEQ ID: NO 7
Human CCBE collagen domain B
GRRGP VGPPG APGRD GSKGE RGAPG PRGSP GPPG
SEQ ID: NO 8
Human CCBE1 collagen domain A
LPGPP GLPGG QGPPG SPGPK GSPGF PGMPG PPGQP GPRGS MGPMG P

1. A protein comprising a fragment of 30-240 amino acids of collagen and calcium-binding EGF domain-containing protein 1 (CCBE1) having 0-10 amino acid substitutions with respect to an amino acid sequence of SEQ ID NO: 1, wherein said fragment comprises at least a collagen domain of CCBE1, or a variant thereof comprising 1, 2 or 3 amino acid substitutions.

2. The protein of claim 1, wherein said collagen domain comprises amino acid 300-333 as defined in SEQ ID:NO 1 or a variant thereof comprising 1, 2 or 3 amino acid substitutions, or an equivalent of said domain in an ortholog of human CCBE1, or a variant thereof comprising 1, 2 or 3 amino acid substitutions.

3. The protein of claim 1, wherein said fragment comprises two collagen domains of CCBE1.

4. The protein of claim 3, wherein said collagen domains comprise amino acid 245-290 as defined in SEQ ID:NO 1 or a variant thereof comprising 1, 2 or 3 amino acid substitutions; and amino acid 300-333 as defined in SEQ ID:NO 1 or a variant thereof comprising 1, 2 or 3 amino acid substitutions, or an equivalent thereof in an ortholog of human CCBE1 or a variant of said equivalent comprising 1, 2 or 3 amino acid substitutions.

5. The protein of claim 1, comprising amino acids 245-333 of human CCBE1 as defined in SEQ ID:NO 1 or a variant thereof comprising 1, 2, 3 or 4 amino acid substitutions, or an equivalent thereof in an ortholog of human CCBE1 or a variant of said equivalent comprising 1, 2, 3 or 4 amino acid substitutions.

6. A nucleic acid molecule encoding the protein of claim 1.

7. A cell or non-human animal comprising the nucleic acid molecule of claim 6.

8. A binding protein that binds the collagen domain of the protein of claim 1.

9. The binding protein of claim 8 wherein the binding protein is an antibody or functional part, derivative or analogue thereof.

10. A method of producing lymphatic channels, increasing lymphangiogenesis and/or inducing a migration of lymphangioblasts in an animal, comprising administrating an effective amount of the protein of claim 1 to said animal.

11. A method of inhibiting a formation of lymphatic channels, lymphangiogenesis and/or a migration of lymphangioblasts in an animal comprising administering an effective amount of the binding protein of claim 8 to said animal.

12. A method for treating cancer in an animal wherein the protein of claim 1 is administered to the animal and produces lymphatic channels, increases lymphangiogenesis and/or induces a migration of lymphangioblasts in the animal.

13. A method for treating cancer in an animal wherein the binding molecule of claim 8 is administered to the animal and inhibits a formation of lymphatic channels, lymphangiogenesis and/or a migration of lymphangioblasts in the animal.

14. The method of claim 12 wherein the animal is a human.

15. A method for manipulating a formation of lymphatic channels, lymphangiogenesis and/or a migration of lymphangioblasts in a cell culture capable thereof, comprising providing the cell culture with the protein of claim 1.

16. The method of claim 13 wherein the animal is a human.


SEQUENCE LISTING (8 sequences)

SEQ ID NO: 1 - protein, 406 aa, Artificial
  Human CCBE1 amino acid sequence
MVPPPPSRGGAARGQLGRSLGPLLLLLALGHTWTYREEPEDGDREICSESKIATTKYPCLKSSGELTTCYRKKCCKGYKFVLGQCIPEDYDVCAEAPCEQQCTDNFGRVLCTCYPGYRYDRERHRKREKPYCLDIDECASSNGTLCAHICINTLGSYRCECREGYIREDDGKTCTRGDKYPNDTGHEKSENMVKAGTCCATCKEFYQMKQTVLQLKQKIALLPNNAADLGKYITGDKVLASNTYLPGPPGLPGGQGPPGSPGPKGSPGFPGMPGPPGQPGPRGSMGPMGPSPDLSHIKQGRRGPVGPPGAPGRDGSKGERGAPGPRGSPGPPGSFDFLLLMLADIRNDITELQEKVFGHRTHSSAEEFPLPQEFPSYPEAMDLGSGDDHPRRTETRDLRAPRDFYP

SEQ ID NO: 2 - DNA, 6276 bp, Artificial
  Human CCBE1 cDNA nucleotide sequence
cggccgcggaggagcaggacgcttggtccggacggagctcggcgctgggaagaagccgggagcttccctgatggtgccgccgcctccgagccggggaggagctgccaggggccagctgggcaggagcctgggtccgctgctgctgctcctggcgttgggacacacgtggacctacagagaggagccggaggacggcgacagagaaatctgctcagagagcaaaatcgcgacgactaaatacccgtgtctgaagtcttcaggcgagctcaccacatgctacaggaaaaagtgctgcaaaggatataaatttgttcttggacaatgcatcccagaagattacgacgtttgtgccgaggctccctgtgaacagcagtgcacggacaactttggccgagtgctgtgtacttgttatccgggataccgatatgaccgggagagacaccggaagcgggagaagccatactgtctggatattgatgagtgtgccagcagcaatgggacgctgtgtgcccacatctgcatcaataccttgggcagctaccgctgcgagtgccgggaaggctacatccgggaagatgatgggaagacatgtaccaggggagacaaatatcccaatgacactggccatgagaagtctgagaacatggtgaaagccggaacttgctgtgccacatgcaaggagttctaccagatgaagcagaccgtgctgcagctgaagcaaaagattgctctgctccccaacaatgcagctgacctgggcaagtatatcactggtgacaaggtgctggcctcaaacacctaccttccaggacctcctggcctgcctgggggccagggccctcccggctcaccaggaccaaagggaagcccaggcttccccggtatgccaggccctcctgggcagcccggcccacggggctcaatgggacccatgggaccatctcctgatctgtcccacattaagcaaggccggaggggccctgtgggtccaccaggggcaccaggaagagatggttctaagggggagagaggagcgcctgggcccagagggtctccaggaccccctggttctttcgacttcctgctacttatgctggctgacatccgcaatgacatcactgagctgcaggaaaaggtgttcgggcaccggactcactcttcagcagaggagttccctttacctcaggaatttcccagctacccagaagccatggacctgggctctggagatgaccatccaagaagaactgagacaagagacttgagagcccccagagacttctacccatagcacatcccaacaccgtcacgccaaaggaagagaaagatcaactcacctgcagttaaaccatctaaagagaagaaagaccactggagacctagaaaacatacatttttctcttctcttctcctgacgtctctccactcctcttcttccaaatacgatgctattttcagagtcccctcctaggcctgcagacatgagggagtgaatgattgatttacctgcttctcactaagagtccattggggtggtttgcattgtaacttttcttttacatcctatttttccaggaactttggatttaagtactctcacagtgtcttaaatcataaattcttgaagttaaatttggcagagtatcaaaagggggaaaatgacaaagtgagctctaagaaaatgtgaggctacttctaagatgtgtgttcacaatagaccataactcctctagtatcaaaattggggctcttcagttaaaaaggggtggggaggacaaacgtgtcgatgtgctttggtggagaattttttccttgtgcttctagtagactttaaatattgtatccctttgtcaaaccttgtttcccaaattcaattaaagagaggagagaattgaatggcgtttagagaagatagaaaagaatcacagtcatatatttactgttatatagattgccacattctaaaattcaaatacggtgcttaaggtttcatgccatgcttatctgtaagtatcctatttagggaagaagattaaactctcttttcaaaaaaacaaagtgaaatgcctggattcacattaaaacaatgggctctcgtttgctataatattttaaagctgtttaatcaacagtggagtctgctctataaatatagattatttgttcaataaactggctgagcttagagagaggtgcagaattcctggttctgagcaggtgcccagaaggtaccattaggtgccatgatccaggctgaaccaatatacagtggggctgaagtctgcaaggaggttgctggcttgggctgacctcactaatgccatcagcagcggtaggtaaattttttctccttgggtattacaagtttttgtctggagccaaccaagcttgccaccaacatattgagagtaatacactattgaaagttatcttggatggggagaaaaaaaaatagtggttttccttgtttgcaaaaacttccttcctattctcattttttcttaattttctttaatttagtccaagttccagttcttttaggccttctctttgatttattttcccctgcatgtgagaagcagttcagaaaaaggtctatatctccacctcctagtgagttagagtgttttctcagagcacctctgggtggcaaagggaagcatgttcctgccaaggtttgctgtggattcagaagcaccaggagcaagagaccagaaggatgatctgctcctttgtaacgttgttgagggccctcttgtttccaatgagcagcttataggttactcacagtccactttctcactggacacacaaagtggctctttatctacctttgcgggagattttcactctcctgcaaatgatcgttctcacactcatattagctcatgttggaatttcccatcctgccatgtcctttcccatttctttttggcttttttgcctccaccttttagcccacatcatttaactccactactgtgaaagcttgcttaaagaaaatccctcttggccgggtgtggtagcccacgcctctaatcccagcactttgggaggctgaggcggggagatcacaaggtcaggagatcgagaccagcctgaccaacatggtgaaaccctgtctctactaaaaatacaaaaattagctgggcgtgttggcacacacctgtaatcccagctactcaggaggctgaggcaggagaattactttaacctgcggggggagcctagattgcgctactgcactccagcctaggcaacagagggagactctgtctcattaaaaaaaaaaaaaaaaaaaaaaaaaggccgggcttggtggctcatgcctgtaatcccagcactttgggaggccgaggcgggcggatcacgaggtcaggagatcgagaccacagtgaaaccctgtctctactaaaaacacaaaaaattagtcgggcgtggtggtgggcgcctgtggtcccagctgctcgggaggctgaggcaggagaatggcgtgaacctgggaggcggagcttgcgctgagccgagatcgtgccactgcactccagcctgggcgacagagcgagactctgtctcaaaaaaaaaaaaatatcccttttgttgtgtttgaatgccacctttcagtcttcttccttaagaatgttaggaaggggctttgcaaacctctagtggcaacagtcttccttcccttcttcctaccacgttggaaatgcaggactcatgtcagtcagcatgtgaccgagtctttcatgaacactttgctcctctatgtctcatttatgcaaaattcctggtgactctccactaacatttttaaagtgaaagcaaactttcactttaaaaaaagtgagcgagaggatcccttgagcccaggagttcaaggccagtctgggcaacatagcaagaccccatccctaaaaaaaaaaaaataagttttattaaatgtggcactttcccctagaagggcagtaaccccaggaaccagtctgcctcttttcgaataaaggagactttttaaaaagaaaaaatttctttttagggtggggtgctctttatctagcatctttccttcctccttctctctgcaaaattcaacattcatgacagagggccacaccaaccacgggtttatagagagaggaactgaacagctgttgattcccatctggaatatatttaatgtttgcctgcttgtggagcatggtatttaaagggtgtggatggatgaatgtttaaaaaaaaacacacaaaactatgcacaagctgtatgagagatggctaggtccatctatgattttttttaaaagaagaattttaataccttttctttgacagtgaactgtatttagaaatatgatctttattgtgaaatgctcataaaatgcaaccaaactgcatgtttggccataggctagaataatatattaaaagaattaatggaaagtagtattgtctgggtttcatgaaactattccagataatattctccagcaaaacttatggtaacctgtattttataatgtcaatctgctggaccaagttaatttagaaaataatttttaatcctcagattttcaaaacttggactactgcaagactgatatgtactttttatgcccaaatcatatttttcaaagataggacatcataaggcatcattatttctaatgtatcttttaactagatagaaaaagctttactgtctaaacaggacttctgctcaaaacctaatgggaaagctatttctggggtaagatttgtaacagtgcttctaggtcatttcatgttttatagggaaaaagatctttgtacacctcctcaacctgttagtagaaatgttgacttcgattttttgcaccctcttcacaggacctcataagttgcctctgttcttggaaagcaatgggaataaaaatatgctgttgaaaatagtgcatggggccaggcacagtggttatgcctgtaatcccaacactttgggaggctgaggcgggcgcatcatgaggtcaggagatcaagaccatcctgaccaacacgtggtgaaaccccatctctactaaaataaataaaaaaaaaaattagccaggcgtggtggtgcacgcctgtagtctcagctactcgggaagctgagacacaagaattgcttgaacccgggaggtggaggttgcagtgagccaagattgcgccactgcactccagcctggctacagagtgagactccgtctcgaaaaaagaaaatagtacatggagggcatgcattttagtttaggaaacccccctcccttcccagttccagagggagaaattataagctatactgtgccaaaagtgagtggggctgcagagagaatggtgccttttctcaccttctgagttaatgcccgacatctgccattggattcattgaccaaaggaagtcatttctactgtataaactcaggagtatcatagcccaacaagtaaataataaatgttcattggttttggaaggaaggaaaggtgagtattaccccaccaaataccagcgaagagcaccctggctttggccatctttccaaggttcataattgctgtgggcctggtgagccctgttcccaggtatgcaagggagggcttctccaggccagacatttctgtaggttttcaggtagcaactcccccatcatattttgggtgtcattccccactctctctccccacttctcagcctgtgccccagaaacaaccacagcagcctcattctgcagacttaacatctgtcaaataaccttcctgaaaaggaaagttattggaggaatccaggccaaacaacaagtagaatagagccatctaaagttattaataatgagttcagggaaatagatgaattttttcctggcatagtcaatatttatatctcacaacaagtcaaaaataattttccatttggaaaacaaaacacaccatgactgtaattgttaaggcatataatcatgaacttcacatgctccctcaacaaacgttactgctctagggaaatcatagaaaaggaccctcttactccccaggggatcaaagcaagtccccttaggaatttcatgtgtgccgtgggtctgtttttatttcacagtgactagtcatatactggagtagctctgttgttcattatttgtaacaggtccatgtaacagtcaagttagcattcaagcaattatggatgtgatactatgatgtacttttgttatgattgtatatgcttaataacaaagttatttttcttaaaaaaaaaaaaaaaaa

SEQ ID NO: 3 - protein, 408 aa, Artificial [sequence omitted]

SEQ ID NO: 4 - DNA, 5742 bp, Artificial
  Mus musculus CCBE1 cDNA nucleotide sequence
gaaggcggctgcggccgcggagcctccctgggcacagcgcagggagagagctcgggcgccgagaagaagccgcgagcttccccgatggtgccgccgcctcttccgagccggggaggagcagccaagaggcagctgggcaagagcctggggccgctgctgctactcctggcgctgggacacacgtggacctaccgagaggaacccgaggaccgcgacagagaagtgtgctctgaaaacaagatcaccacgaccaaatacccatgtctgaagtcttcgggcgagctcaccacgtgcttcaggaaaaaatgctgcaaaggatataaatttgttcttggacagtgtattccagaagattatgacatttgtgcccaagctccctgtgaacagcagtgcacggataacttcggccgtgtgctgtgcacgtgctacccgggataccgatatgaccgcgagagacaccaaaagcgggagagaccgtactgtctggatatcgatgaatgtgccaccagcaataccactctctgtgcacatatctgcatcaacaccatgggcagctaccactgtgagtgtcgggagggctacatccttgaggacgatgggaggacctgcacaaggggagacaaataccccaatgacactgggcatgaagagaagtccgagaatgaggtgaaagccgggacctgctgcgccacatgcaaagagttctcccagatgaagcagaccgtgctgcagctgaaacagaagatggcgctgctccccaacaatgccgctgaactgggcaagtatgtcaatggtgacaaggtactggcctcaaacgcctaccttccaggacctcctggcctgcctgggggacagggcccaccaggctcaccaggacccaaggggagccctggattccccggtatgccaggccctcctggacagccaggcccacggggctcaatgggacctatgggaccttcccctgacctgtctcatattaagcaaggccggaggggccccgtgggcccaccaggagcaccaggaagacacggctccaagggggagagaggagcacccggacccccggggtctccgggacccccaggttctttcgacttcctgctgcttgtgctggcggacattcgaaatgacatcgcagagctgcaggagaaggtgtttggacaccggactcactcttccgccgaggacttccccctacctcaggaattctccagctacccagagacgctggactttggctctggagatgactactccagaagaactgaagcaagggaccccgaggccccaagaaatttttatccatagcacatcccatatcctcacgccaccagaagagaagcatgccttcgaccttccccttactctgtttccaaacccggttttccaagtttcctgtgcatcacactagcaacaggaagatgtgatgggttaacgtgctgttcttgaagagccaatgggtggttgacatcaagactttcttcaacgtccgattttttcaagagcttttgggtttctgagttcatcattccacagaatcttcaactttggcagagtgcaaaagaggggaaaatggcaccgtgggttcttaggagtgtaaggttccttctaaaaagaaaataatgttcaaatgcttgactattcattccctactgtcaggagtaaggcttttagggtgggggtgggggtggaggggcatggaaggagcaaccatggagaataacattgatagaaaatgattttccttagcaatatcccagatttcaaattctgcatcctattttccaacttgatgggggtttttttttgtttgtttgttttggtttggttttttgttttttgggggtgtttgtttgtttgtttgtctttaaatttggttacagaaacaagagattggaaagaatgtgttcagagagatagagaattagccttatatttcctgtctcataggttgccgaatttagaattcagatacgacagtcattgattcgtgcaaatcctatgtggaggagggagtttgtttgtaagggcttgatcatgttttctagggagcttggggtgaggaaacactctgtgcatactagaacagagggctcctggagaccaggacgattgaagactacatggtcaaaagcattgtcctgctccatacatgttgatgatttgtgaaataagatggctgacatttaagaagaagtacaagatgccctggcactgacagggttgccagatgtgccagcaaggcctgatccaggctgaatcaaggctggagggaaatcactgccctggaatcttagccaaggatgtcaaggggacattgagaagtgcttcactaaaatcagattaatcagaagcagattaatctactcttaagagatttgaagttctcaccagggcacacttggaacagcatacagctgagaggtctctgagatagagaaggaggaggagtttttgataaattggaataattttccactatttgccccctgatttggttagaatccctgttgttctgggtgtccttgactttttgtcctgggccacaaggaaacggcttggaggccagcacatttcccaatgtctccctaagtcccttcttggagtatgtctagatccccagggaatcatgttcccagaaaggagcaagaggccagcaggttccttcttggtatctctgggttcttgtttcagggatctgcttccgggctccttagtgaacactttctccttgggtgtgcagcaggctccgtgatcttgctcacatggttgcccacaccagggtcccagtttgctatcctgcagtgcccccttatatttctaagaagtttgagattgcattgatcttttccctatatcacttaagcctccatcgtcttcccacacctactgtgacagcttgttggaggaaacagctattggtttgcatgacactgcatccaccatccactcttctttctgagcaataagaaggggctctgtctacagagccagccagagcctcgtgggctttcacttcccctctcccacctccctaccctcatccctctctcctggttctcttccttttccttttttttctctcttcctctcctcctcttttgctccttctctccttcatttcctctacccctatcctagcttccctgcctttccacctctcctttcttacttgcttccttcctccctccttccctccctccctttctccttctctccctccatctttcttcctttctactccctccctcccttcctacctacctacttccttccttcccttttttcttccttagttccctctctctgctccctccctccttccctccatgcttgcctgcctatcttcctacccagttagaaaggcaggattcccacacactcccacccaaatccccagtggctgtccatcagcagtcctgatgaaggcaagcacacacttgcatcgcttgcttgctgtctcaccctccttcccccttcagtctctcctgaacctgtgcatcaccacagatagccttgtcaactgtggcttcctgaagagatggatgcaaccgcagtcaagtgccacctggaatctatttagtgcttgcctgcttatagaacccatcttttaaagggtgtggatggatgaattctcaaaactatgcacaggctctgtgagcaataattatgtccatctttttttatgaggaattttataactactttttatgtctactttttttagacagtgaaatgtatgtagaaatataatctttattgccaaatgctggtaaaacgcgagcacactccatgcttggctggaggccaggataatgtactgaaagaatgaagagaaagtcataatatctgggattcatgagtctgtctagataattacctccaggaaaacataatagcctatattttgtgatctcaatatgccatgtcaatttagataatcgttactcctcagattttcaaaacatggaatactaactaactacaagaccaacgcatgtcctctacttattcaaatcatggatttttagtgataaagcattattgctcccatatcttttagttgtggacagaaaaaaaaatcaaagcttttctgtctaaacaaatttctgctcaaaatatcataggaaggcctttgggaggaaggacagttcagaacaaatttaccgagtcacttcatatctcacagggaaaatattttagttttttatacgcccttcaacactttagtagaaatggtcaacttttctaatatttccactggttaagacttggttatgaattgcctctgtgtttataaagcaatgagaataaaatttcactgaaaataatataaagaagactttcattttagtttaggaaaactgaattttccaattccagaaggaaaaaaataaccagttttcctgtgccagttaagtgttaggagctaggtgtgatggtggacacctgtaatcccagcacctgagaagcacagctaggcagatcaggagttcaaggtcagcctcagctacataacaagtgtgaggccagcctgggctatctgagagtctatctcaaaataataatgatgatgatgataatgtgatgataataaagaataataattagcagaggactaaagagatggctcagaggttaaggattcttgctgctcttccagagatacagagctcagtttccagctcttgcacaggcagctcacaacatctgtaaacacaggtccaggtgatctggcgccctcttctggcttccatagtacctgcactcacatgcacacacacacatagatatacaaacacataattacaaataaagtcttttttaaaggagtgggtacaaagaggatgctgccttttcttgctttctatgttactttccaatacttgctacggaattaactaaagtcatattgctgtgtggagccaggccactatccagcaggaaaattataagtgcccagtgatcttggaagaagttttggcccatcaaaggccaggtgcagagcaccctggtagacccaggtaagtcatgttccctttccaaggttctgaacatccttggccttggaagtaccactatcaggcacattggcacaaaacttcttcatgaccatccatcaaactttaccttgaggtctcaggaagccacccccctatcttattttgagcatcatttttcattttcctcctcttccaaattcaaacagacagcctcaggagcctccttctgcaagcttatcttccagacaactgttctaaaagggaaagctattggctggaatctgagccatacaattagtaatatagaggcttctgttctattcagggaaatacatgggtcatttcctgatgcagcaatcatgtatgtttcacaacatgtcaaaaataattttctctttggatttttcaagggccgtcattgttaaagctgaaatggtgtgctttatatactcttcctacaaatgttaccatcagagaggaaccatgaaaaaggacatatgggctatccatccctcagagagctcagtatgttttgtccgattcccagagcttgatccctgagtggttcttgagtctgtttttatttctcagtcactggtcatatatttcagtatctctgtgcattatttatatcagatcttgtaacagtcaaactgatattccaacagtcatggatgtaatactaggaagtgcgtttgtatgactgtatatgcttaataacaaagttatttttctt

SEQ ID NO: 5 - protein, 401 aa, Artificial
  Danio rerio CCBE1 amino acid sequence
MIYPGRGASLSVAVALVLFSSGAPWTFREEKEDVDREVCSESKIATTKYPCVKSTGEVTTCYRKKCCEGFKFVLGQCIPEDYDVCAGAPCEQQCTDHFGRVVCTCYDGYRYDRERHRNREKPYCLDIDECANNNETVCSQMCVNTPGSYRCDCHSGFYLEDDGKTCTKGERAPLFEKSDNVMKEGTCSATCEDFHQMKMTVLQLKQKMSLLSSNTEINKQMTNEKMMMTTNSFLPGPPGPPGPAGTPGAKGSSGSPGQMGPPGLPGPRGDMGPIGPSPDLSHIKQGRRGPVGPPGAPGRDGMKGERGFPGPSGPPGPPGSFDFLLLMMADIRNDIAELQSKVFSRPLHSSFEDFPSAPDSWRDTPENLDFGSGEDYKSQSPPKSSRKRKLPRNLKNPDWPV

SEQ ID NO: 6 - DNA, 1517 bp, Artificial
  Danio rerio CCBE1 partial cDNA nucleotide sequence
ggaatctgcgcgtggggcttcgcaggaggtaaccaatcaatactctcccaattcctccgaaacaagcgctgaacttcaagactggatttaaagggacaaatatcttttaatcatctcagagtgtctgaaatgatctacccgggcagaggagcatctctcagtgtggccgtggcgctcgtactgttctcatcgggagctccgtggactttccgagaggagaaggaggatgtagacagagaagtgtgttcagaaagcaaaatcgcaacgacgaagtacccgtgcgtaaagtccactggagaggtgaccacatgctacagaaagaagtgctgtgaaggtttcaagtttgtcctgggtcagtgtattccagaagattatgatgtgtgtgccggcgccccgtgtgaacagcagtgtacggatcatttcggtcgggttgtttgcacctgctatgacgggtatcgatatgaccgggaacgacacagaaacagggagaaaccatattgcctggacattgatgaatgtgccaacaataacgagactgtgtgctcacagatgtgtgtgaacactcccggcagctaccggtgcgactgccacagcggcttctacctggaggatgatgggaagacatgcaccaagggcgagagagctccactctttgaaaaatcagacaacgtcatgaaagaaggcacgtgttcggccacatgcgaggacttccatcagatgaagatgactgttctccaactcaaacagaagatgtccttattgtcgagcaacaccgagatcaataagcagatgaccaatgaaaaaatgatgatgacgacaaattcatttttacctggacctccagggcctcctgggcctgcagggacgcctggagccaaaggatcctctggctctcctggacagatggggccaccaggcttacctggtcccagaggtgacatgggacccattggaccctcccctgacctgtcgcatatcaaacagggtcgacgtggcccagtgggtcctcctggggctccagggagagatggcatgaagggagagcgagggtttcccggcccttccggacctccaggcccaccaggatcctttgacttcctgttactcatgatggctgacattcgtaacgacatcgctgagctgcagagtaaagtcttcagcagaccgttacattcttcatttgaggatttcccatctgccccggacagctggagagacacaccggagaacctggacttcggatccggcgaagactacaaatcccagagccctcccaagagcagcaggaagaggaaattacctcggaacttaaaaaatcccgattggccggtttgagactgtcagtaaacaatacaagaactgaaaaaactgatggtgtaaatattacagaatgactgctatttaactgtatactcgctgaggaaagaaggctgaatatatttcactgtatgaaatttctatttttataacataaaagatgtacattttttgtaaaaaaaaaaaaaaaaaaaaaaaaa

SEQ ID NO: 7 - protein, 34 aa, Artificial
  Human CCBE collagen domain B
GRRGPVGPPGAPGRDGSKGERGAPGPRGSPGPPG

SEQ ID NO: 8 - protein, 46 aa, Artificial
  Human CCBE1 collagen domain A
LPGPPGLPGGQGPPGSPGPKGSPGFPGMPGPPGQPGPRGSMGPMGP